SEQUENCE LISTING (42 sequences)

SEQ ID NO: 1 - DNA, 4679 bp, Adeno-associated virus type 2
  source 1..4679: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 480 bp, synthetic construct
  source 1..480: mol_type = other DNA
tgaccgcgaaccagtttggcaaagtcggctcgatcccgcggtaaattctctgaatcagtttttcgcgaatctgactcaggaaacgtcccaaaaccatggatttcaccccggtggtttccacgagcacgtgcatgtggaagtagctctctcccttctcaaattgcacaaagaaaagagcctccggggccttactcacacggcgccattccgtcagaaagtcgcgctgcagcttctcggccacggtcaggggtgcctgctcaatcagattcagatccatgtcagaatctggcggcaactcccattccttctcggccacccagttcacaaagctgtcagaaatgccgggcagatgctcgtcaaggtcgctggggaccttaatcacaatctcgtaaaaccccggcataggaacccctagtgatggagttggccactccctctctgcgcgctcgctcgctcactgaggccgggcgaccaaaggtc

SEQ ID NO: 3 - DNA, 480 bp, synthetic construct
  source 1..480: mol_type = other DNA
gggcgacctttggtcgcccggcctcagtgagcgagcgagcgcgcagagagggagtggccaactccatcactaggggttccttctagacaactttgtatagaaaagttgggaggaaatggagaccctgcgtgctcactcgggcttaaatacccagcgtgaccacatggtgtcgcaaaatgtcgcaaaacactcacgtgacctctaatacaggacctctagtcctgcaggtttaaacgaattcgcccttcgcagagaccaaagttcaactgaaacgaatcaaccggtttattgattaacaggcaattacagattacgagtcaggtatctggtgccaatggggcgaggctctgaatacacgccattagtgtccacagtaaagtccacattaacagacttgttgtagttggaagtgtactgaatttcgggattccagcgtttgctgttttccttctgcagctcccactcgatctccacgctgac

SEQ ID NO: 4 - DNA, 21 bp, synthetic construct
  source 1..21: mol_type = other DNA
agtggccaactccatcactag

SEQ ID NO: 5 - DNA, 21 bp, synthetic construct
  source 1..21: mol_type = other DNA
agtggccaactccatcactag

SEQ ID NO: 6 - DNA, 19 bp, synthetic construct
  source 1..19: mol_type = other DNA
gccacccagttcacaaagc

SEQ ID NO: 7 - DNA, 22 bp, synthetic construct
  source 1..22: mol_type = other DNA
gtggacactaatggcgtgtatt

SEQ ID NO: 8 - DNA, 22 bp, synthetic construct
  source 1..22: mol_type = other DNA
gagcttccaccactgtcttatt

SEQ ID NO: 9 - DNA, 23 bp, synthetic construct
  source 1..23: mol_type = other DNA
ggatttggatgactgcatctttg

SEQ ID NO: 10 - DNA, 27 bp, synthetic construct
  source 1..27: mol_type = other DNA
agcgaccttgacgagcatctgcccggc

SEQ ID NO: 11 - DNA, 24 bp, synthetic construct
  source 1..24: mol_type = other DNA
cccattggcaccagatacctgact

SEQ ID NO: 12 - DNA, 24 bp, synthetic construct
  source 1..24: mol_type = other DNA
tggaagataaccatcggcagccat

SEQ ID NO: 13 - DNA, 296 bp, synthetic construct
  source 1..296: mol_type = other DNA
agtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggc

SEQ ID NO: 14 - DNA, 122 bp, synthetic construct
  source 1..122: mol_type = other DNA
ggatttggatgactgcatctttgaacaataaatgatttaaatcaggtatggctgccgatggttatcttccagattggctcgaggacactctctctgaaggaataagacagtggtggaagctc

SEQ ID NO: 15 - DNA, 222 bp, synthetic construct
  source 1..222: mol_type = other DNA
gtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccact

SEQ ID NO: 16 - DNA, 21 bp, synthetic construct
  source 1..21: mol_type = other DNA
agtggccaactccatcactag

SEQ ID NO: 17 - DNA, 19 bp, synthetic construct
  source 1..19: mol_type = other DNA
gccacccagttcacaaagc

SEQ ID NO: 18 - DNA, 27 bp, synthetic construct
  source 1..27: mol_type = other DNA
agcgaccttgacgagcatctgcccggc

SEQ ID NO: 19 - DNA, 22 bp, synthetic construct
  source 1..22: mol_type = other DNA
gtggacactaatggcgtgtatt

SEQ ID NO: 20 - DNA, 21 bp, synthetic construct
  source 1..21: mol_type = other DNA
agtggccaactccatcactag

SEQ ID NO: 21 - DNA, 24 bp, synthetic construct
  source 1..24: mol_type = other DNA
cccattggcaccagatacctgact

SEQ ID NO: 22 - DNA, 22 bp, synthetic construct
  source 1..22: mol_type = other DNA
gagcttccaccactgtcttatt

SEQ ID NO: 23 - DNA, 23 bp, synthetic construct
  source 1..23: mol_type = other DNA
ggatttggatgactgcatctttg

SEQ ID NO: 24 - DNA, 24 bp, synthetic construct
  source 1..24: mol_type = other DNA
tggaagataaccatcggcagccat

SEQ ID NO: 25 - DNA, 21 bp, synthetic construct
  source 1..21: mol_type = other DNA
gggaatacagaaccaccagac

SEQ ID NO: 26 - DNA, 21 bp, synthetic construct
  source 1..21: mol_type = other DNA
agtggccaactccatcactag

SEQ ID NO: 27 - DNA, 23 bp, synthetic construct
  source 1..23: mol_type = other DNA
tcggaacccgataccttacccga

SEQ ID NO: 28 - DNA, 23 bp, synthetic construct
  source 1..23: mol_type = other DNA
ggatttggatgactgcatctttg

SEQ ID NO: 29 - DNA, 21 bp, synthetic construct
  source 1..21: mol_type = other DNA
gaccttcaccaacttcttcca

SEQ ID NO: 30 - DNA, 24 bp, synthetic construct
  source 1..24: mol_type = other DNA
ttgttgatcaccctccagattggt

SEQ ID NO: 31 - DNA, 22 bp, synthetic construct
  source 1..22: mol_type = other DNA
tctacaagtgtggactttgctg

SEQ ID NO: 32 - DNA, 21 bp, synthetic construct
  source 1..21: mol_type = other DNA
agtggccaactccatcactag

SEQ ID NO: 33 - DNA, 30 bp, synthetic construct
  source 1..30: mol_type = other DNA
aggcgtgtactctgaaccccgccccattgg

SEQ ID NO: 34 - DNA, 23 bp, synthetic construct
  source 1..23: mol_type = other DNA
ggatttggatgactgcatctttg

SEQ ID NO: 35 - DNA, 18 bp, synthetic construct
  source 1..18: mol_type = other DNA
ggggctccaggtttcagc

SEQ ID NO: 36 - DNA, 24 bp, synthetic construct
  source 1..24: mol_type = other DNA
tggaagataaccatcggcagccat

SEQ ID NO: 37 - DNA, 24 bp, synthetic construct
  source 1..24: mol_type = other DNA
tgaaggtgtatatagtgaaccccg

SEQ ID NO: 38 - DNA, 21 bp, synthetic construct
  source 1..21: mol_type = other DNA
agtggccaactccatcactag

SEQ ID NO: 39 - DNA, 24 bp, synthetic construct
  source 1..24: mol_type = other DNA
cccattggcaccagatacctgact

SEQ ID NO: 40 - DNA, 23 bp, synthetic construct
  source 1..23: mol_type = other DNA
ggacttggatgactgtgtttctg

SEQ ID NO: 41 - DNA, 20 bp, synthetic construct
  source 1..20: mol_type = other DNA
caccactcgcgaattccttc

SEQ ID NO: 42 - DNA, 24 bp, synthetic construct
  source 1..24: mol_type = other DNA
tggaagataaccatcggcagccat

(19) United States
(12) Patent Application Publication
(10) Pub. No.: US 2026/0193723 A1
(43) Pub. Date: Jul. 9, 2026

(54) METHODS OF DETECTING REPLICATION COMPETENT AAV

(71) Applicant: SparingVision SAS, Paris (FR)

(72) Inventors: Mehdi GASMI, Palavas-les-Flots (GR); Fabien DORANGE, Nogent-le-Phaye (FR)

(21) Appl. No.: 19/012,333

(22) Filed: Jan. 7, 2025

Related U.S. Application Data
(60) Provisional application No. 63/709,118, filed on Oct. 18, 2024.

(51) Int. Cl. C12Q 1/70; C12Q 1/6851

(52) U.S. Cl. CPC C12Q 1/701; C12Q 1/6851

(57) ABSTRACT

Provided herein are methods of detecting and quantifying replication competent adeno-associated virus (rcAAV) in a recombinant adeno-associated virus (rAAV) sample comprising multiplex digital PCR (dPCR). Also provided herein are methods of performing quality control on an rAAV product.

### RELATED APPLICATIONS

This application claims the benefit of U.S. Provisional Application Ser. No. 63/709,118 filed Oct. 18, 2024, which is hereby incorporated by reference in its entirety.

### SEQUENCE LISTING

This application contains references to amino acid sequences and/or nucleic acid sequences which have been submitted concurrently herewith as the sequence listing xml file entitled “115738_833928_SL REPLACEMENT 2.xml”, file size 44,302 bytes, created on Apr. 11, 2025. The aforementioned sequence listing is hereby incorporated by reference in its entirety pursuant to 37 C.F.R. § 1.52 (e) (5).

### FIELD

The present disclosure relates in some aspects to methods for detecting replication competent adeno-associated virus (rcAAV) in a recombinant adeno-associated virus (rAAV) sample.

### BACKGROUND

Gene therapy treatments based on adeno-associated virus (AAV) vectors offer significant clinical potential. However, during the production of AAVs, contaminants such as replication competent AAVs (rcAAVs), may form and pose a significant risk to the safety of AAV therapies. rcAAVs are formed from the recombination of ITR-bearing DNAs with rep and cap sequences. Currently, methods to detect and quantify rcAAV fail to provide sensitivities needed to ensure the safety of rAAV therapeutics. For example, current cell-based assays for rcAAV detection display biases depending on the AAV serotype and cell line used in the assay. These biases severely limit the sensitivity of cell-based assays. Thus, improved methods for detecting and quantifying rcAAV during the production of rAAV products are needed. Provided herein are methods that address such and other needs.

### BRIEF SUMMARY

Provided herein are methods of detecting replication competent adeno-associated virus (rcAAV) in a recombinant adeno-associated virus (rAAV) sample, comprising at least one multiplex digital polymerase chain reaction (dPCR) amplifying at least three junctions of a rcAAV genome comprising a first junction of an rcAAV genome, a second junction of an rcAAV genome, and a third junction of an rcAAV genome in the rAAV sample, wherein the rcAAV comprises a contiguous 5′ITR, Rep gene, Cap gene, and 3′ITR, and wherein said first junction comprises a nucleotide sequence comprising the 3′ end of the 5′ITR and the 5′ end of the Rep gene; said second junction comprises a nucleotide sequence comprising the 3′ end of the Rep gene and the 5′ end of the Cap gene; said third junction comprises a nucleotide sequence comprising the 3′ end of the Cap gene and the 5′ end of the 3′ITR.

Also provided herein, in some aspects, are methods of detecting replication competent adeno-associated virus (rcAAV) in a recombinant adeno-associated virus (rAAV) sample, comprising a multiplex digital polymerase chain reaction (dPCR) comprising; (a) a first dPCR assay comprising the steps of: (i) serially diluting the rAAV sample; (ii) determining the level of each of a first junction of an rcAAV genome, a second junction of an rcAAV genome, and a third junction of an rcAAV genome in the rAAV sample by dPCR; (iii) identifying an optimal dilution for single dPCR quantification of rcAAV in the rAAV sample; and (b) a second dPCR assay comprising the steps of: (i) diluting the rAAV sample according to the optimal dilution for single dPCR quantification identified in step (a); (ii) determining the level of each of the first junction of the rcAAV genome, the second junction of the rcAAV genome, and the third junction of the rcAAV genome in the rAAV sample by dPCR; (iii) detecting rcAAV in the rAAV sample when the first junction of the rcAAV genome, the second junction of the rcAAV genome, and the third junction of the rcAAV genome in the rAAV sample are detected within a single partition, wherein the rcAAV comprises a contiguous 5′ITR, Rep gene, Cap gene, and 3′ITR, and wherein said first junction comprises a nucleotide sequence comprising the 3′ end of the 5′ITR and the 5′ end of the Rep gene; said second junction comprises a nucleotide sequence comprising the 3′ end of the Rep gene and the 5′ end of the Cap gene; said third junction comprises a nucleotide sequence comprising the 3′ end of the Cap gene and the 5′ end of the 3′ITR.

In some aspects, the optimal dilution for single dPCR quantification of rcAAV in the rAAV sample is selected as the largest dilution of the rAAV sample performed in the first dPCR assay satisfying the following criteria; (a) the number of triple positive partitions comprises less than about 0.1% of particles, and (b) the number of double positive partitions comprises less than about 1% of particles.

In some aspects, a minimum number of runs to be employed in the second dPCR assay is determined using the formula:

$$Minimum number of runs = \frac{[ 100 * \frac{\# partitions obtained for a dilution during assay 1}{\begin{matrix}\# positive partitions obtained for a \\ dilution during assay 1\end{matrix}} ]}{maximum \# partitions per well} .$$

In some aspects, the first junction of the rcAAV genome is amplified using forward/reverse primers capable of hybridizing to nucleotides 117-412 of the AAV2 genome set forth in SEQ ID NO:13. In other aspects, the first junction of the rcAAV genome is amplified using a forward primer comprising a nucleotide sequence at least 80% identical to the nucleotide sequence set forth in SEQ ID NO:4, and a reverse primer comprising a nucleotide sequence at least 80% identical to the nucleotide sequence set forth in SEQ ID NO:6. In other aspects, the first junction of the rcAAV genome is amplified using a forward primer comprising the nucleotide sequence set forth in SEQ ID NO:4, and a reverse primer comprising the nucleotide sequence set forth in SEQ ID NO:6.

In some aspects, the second junction of the rcAAV genome is amplified using forward/reverse primers capable of hybridizing to nucleotides 2156-2277 of the AAV2 genome set forth in SEQ ID NO:14. In other aspects, the second junction of the rcAAV genome is amplified using: 1) a forward primer comprising a nucleotide sequence at least 80% identical to the nucleotide sequence set forth in SEQ ID NO:8, and a reverse primer comprising a nucleotide sequence at least 80% identical to the nucleotide sequence set forth in SEQ ID NO:9, 2) a forward primer comprising a nucleotide sequence at least 80% identical to the nucleotide sequence set forth in SEQ ID NO:22, and a reverse primer comprising a nucleotide sequence at least 80% identical to the nucleotide sequence set forth in SEQ ID NO:23, 3) a forward primer comprising a nucleotide sequence at least 80% identical to the nucleotide sequence set forth in SEQ ID NO:28, and a reverse primer comprising a nucleotide sequence at least 80% identical to the nucleotide sequence set forth in SEQ ID NO:29, 4) a forward primer comprising a nucleotide sequence at least 80% identical to the nucleotide sequence set forth in SEQ ID NO:34, and a reverse primer comprising a nucleotide sequence at least 80% identical to the nucleotide sequence set forth in SEQ ID NO:35, or 5) a forward primer comprising a nucleotide sequence at least 80% identical to the nucleotide sequence set forth in SEQ ID NO:40, and a reverse primer comprising a nucleotide sequence at least 80% identical to the nucleotide sequence set forth in SEQ ID NO:41. In some aspects, the second junction of the rcAAV genome is amplified using a forward primer comprising a nucleotide sequence at least 80% identical to the nucleotide sequence set forth in SEQ ID NO:8, and a reverse primer comprising a nucleotide sequence at least 80% identical to the nucleotide sequence set forth in SEQ ID NO:9. In other aspects, the second junction of the rcAAV genome is amplified using a forward primer comprising the nucleotide sequence set forth in SEQ ID NO:8, and a reverse primer comprising the nucleotide sequence set forth in SEQ ID NO:9.

In some aspects, the third junction of the rcAAV genome is amplified using forward/reverse primers capable of hybridizing to nucleotides 4342-4563 of the AAV2 genome set forth in SEQ ID NO:15. In other aspects, the third junction of the rcAAV genome is amplified using: 1) a forward primer comprising a nucleotide sequence at least 80% identical to the nucleotide sequence set forth in SEQ ID NO:7, and a reverse primer comprising a nucleotide sequence at least 80% identical to the nucleotide sequence set forth in SEQ ID NO:5, 2) a forward primer comprising a nucleotide sequence at least 80% identical to the nucleotide sequence set forth in SEQ ID NO: 19, and a reverse primer comprising a nucleotide sequence at least 80% identical to the nucleotide sequence set forth in SEQ ID NO:20, 3) a forward primer comprising a nucleotide sequence at least 80% identical to the nucleotide sequence set forth in SEQ ID NO:25, and a reverse primer comprising a nucleotide sequence at least 80% identical to the nucleotide sequence set forth in SEQ ID NO:26, 4) a forward primer comprising a nucleotide sequence at least 80% identical to the nucleotide sequence set forth in SEQ ID NO:31, and a reverse primer comprising a nucleotide sequence at least 80% identical to the nucleotide sequence set forth in SEQ ID NO:32, or 5) a forward primer comprising a nucleotide sequence at least 80% identical to the nucleotide sequence set forth in SEQ ID NO:37, and a reverse primer comprising a nucleotide sequence at least 80% identical to the nucleotide sequence set forth in SEQ ID NO:38. In other aspects, the third junction of the rcAAV genome is amplified using a forward primer comprising a nucleotide sequence at least 80% identical to the nucleotide sequence set forth in SEQ ID NO:7, and a reverse primer comprising a nucleotide sequence at least 80% identical to the nucleotide sequence set forth in SEQ ID NO:5. In other aspects, the third junction of the rcAAV genome is amplified using a forward primer comprising the nucleotide sequence set forth in SEQ ID NO:7, and a reverse primer comprising the nucleotide sequence set forth in SEQ ID NO:5.

In some aspects, the first junction of the rcAAV genome, the second junction of the rcAAV genome, and the third junction of the rcAAV genome are detected in the first dPCR assay and the second dPCR assay using fluorescently labeled hydrolysis probes. In some aspects, the fluorescently labeled hydrolysis probes comprise a fluorescent label selected from the group consisting of 6-Carboxyfluorescein (FAM), Tetrachlorofluorescein (TET), Hexachlorofluorescein (HEX), and Carboxytetramethyl-rhodamine (TAMRA).

In some aspects, the first junction of the rcAAV genome is detected using a fluorescently labeled hydrolysis probe comprising the nucleotide sequence set forth in SEQ ID NO: 10 or SEQ ID NO:18. In other aspects, the first junction of the rcAAV genome is detected using a fluorescently labeled hydrolysis probe comprising the nucleotide sequence set forth in SEQ ID NO:10.

In some aspects, the second junction of the rcAAV genome is detected using a fluorescently labeled hydrolysis probe comprising the nucleotide sequence set forth in SEQ ID NO: 12, SEQ ID NO:24, SEQ ID NO:30, SEQ ID NO:36, or SEQ ID NO:42. In other aspects, the second junction of the rcAAV genome is detected using a fluorescently labeled hydrolysis probe comprising the nucleotide sequence set forth in SEQ ID NO:12.

In some aspects, the third junction of the rcAAV genome is detected using a fluorescently labeled hydrolysis probe comprising the nucleotide sequence set forth in SEQ ID NO: 11, SEQ ID NO:21, SEQ ID NO:27, SEQ ID NO:33, or SEQ ID NO:39. In other aspects, the third junction of the rcAAV genome is detected using a fluorescently labeled hydrolysis probe comprising the nucleotide sequence set forth in SEQ ID NO:11.

Also provided herein, in some aspects, are methods of producing a recombinant adeno-associated virus (rAAV), the method comprising a) recombinantly producing the AAV in a culture media comprising an AAV host cell; b) collecting the rAAV from the media of step (a); and c) quantifying the amount of rcAAV contaminants in the sample using any of the methods described herein; and d) discarding the produced AAV when rcAAV is detectibly present, or not-discarding the produced AAV when rcAAV is not detectibly present or is detectable at a threshold.

Also provided herein, in some aspects, are methods for performing quality control on a recombinant adeno-associated virus (rAAV) product, comprising: a) producing the rAAV product; b) performing a first dPCR assay comprising the steps of: (i) serially diluting the rAAV sample; (ii) determining the level of each of a first junction of an rcAAV genome, a second junction of an rcAAV genome, and a third junction of an rcAAV genome in the rAAV sample by dPCR; (iii) identifying an optimal dilution for single dPCR quantification of rcAAV in the rAAV sample; and c) performing a second dPCR assay comprising the steps of: (i) diluting the rAAV sample according to the optimal dilution for single dPCR quantification identified in step (b); (ii) determining the level of each of the first junction of the rcAAV genome, the second junction of the rcAAV genome, and the third junction of the rcAAV genome in the rAAV sample by dPCR; (iii) detecting rcAAV in the rAAV sample when the first junction of the rcAAV genome, the second junction of the rcAAV genome, and the third junction of the rcAAV genome in the rAAV sample are detected within a single partition, d) discarding the produced rAAV product when rcAAV is detectibly present or detectable above a threshold, or not-discarding the produced AAV when rcAAV is not detectibly present or detectable below a threshold; and wherein the rcAAV comprises a contiguous 5′ITR, Rep gene, Cap gene, and 3′ITR, and wherein said first junction comprises a nucleotide sequence comprising the 3′ end of the 5′ITR and the 5′ end of the Rep gene; said second junction comprises a nucleotide sequence comprising the 3′ end of the Rep gene and the 5′ end of the Cap gene; said third junction comprises a nucleotide sequence comprising the 3′ end of the Cap gene and the 5′ end of the 3′ITR.

Also provided herein are kits for detecting rcAAV particles, wherein the rcAAV particles comprise a contiguous 5′ITR, Rep gene, Cap gene, and 3′ITR, the kit comprising: a) forward and reverse primers capable of hybridizing to a first junction of an rcAAV genome comprising the 3′ end of the 5′ITR and the 5′ end of the Rep gene; b) forward and reverse primers capable of hybridizing to a second junction comprising the 3′ end of the Rep gene and the 5′ end of the Cap gene; c) forward and reverse primers capable of hybridizing to a third junction comprising the comprising the 3′ end of the Cap gene and the 5′ end of the 3′ITR; and d) fluorescently labeled hydrolysis probes capable of hybridizing to the first junction, the second junction, and the third junction of the rcAAV genome.

### BRIEF DESCRIPTION OF THE DRAWINGS

FIG. 1A is a schematic diagram depicting three plasmids commonly used during rAAV manufacturing; a first plasmid comprising a transgene expression construct, a second plasmid comprising AAV Rep and Cap genes, and a third plasmid comprising helper genes. A recombination event between a RepCap plasmid and a transgene expression plasmid may result in the formation of rcAAV.

FIG. 1B is a schematic diagram depicting a representative therapeutic rAAV vector and an rcAAV positive particle.

FIG. 2 is a schematic diagram depicting a representative rcAAV genome. The 5′ITR, Rep, Cap, and 3′ ITR nucleotide domains of the rcAAV genome are shown. Nucleotide sequences targeted by the dPCR assays described herein are indicated as junction 1, junction 2, and junction 3. Regions of the rcAAV genome depicted between brackets denote variable nucleotide sequences and sizes due to recombination events between rAAV and Rep/Cap plasmids. Arrows denote dPCR primers. Fluorescently labeled hydrolysis probes are denoted by a line and star.

FIG. 3 is a schematic diagram showing an exemplary workflow for detecting rcAAV within an rAAV sample.

FIG. 4 is a bar graph depicting the number of rcAAV genomes per 1×109 viral genomes (vg) for a variety of rAAV samples. Shown from left to right, the rAAV samples tested are rAAV-Transgene 1 (rAAV-T1) 250L, rAAV-T1 50L, rAAV-T1 10L, rAAV-T1 10L 2×10L, rAAV-T1 comprising synthetic DNA, rAAV-T1 comprising synthetic DNA (AAV2/8), wtAAV2/2x, wtAAV8, rAAV-Transgene 3 (rAAV-T3) (AAV2/8), rAAV-T1 (AAV2/8) 2×1L, rAAV-Transgene 2 (rAAV-T2) 10L, and rAAV-T3 (AAV2/9).

FIG. 5A is a bar graph depicting results from an AAV2/2 cell-based assay in which cells were spiked with representative numbers of rcAAV genomes, as determined from the dPCR assay described herein. The dashed line represents the limit of detection for the cell-based assay considering the number of triple positive genomes in the positive control batch.

FIG. 5B is a bar graph depicting the results from an AAV2/8 cell-based assay in which cells were spiked with representative numbers of rcAAV genomes, as determined from the dPCR assay described herein. The dashed line represents the limit of detection for the cell-based assay considering the number of triple positive genomes in the positive control batch.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods and kits for detecting replication competent adeno-associated virus (rcAAV) in recombinant adeno-associated virus (rAAV) samples. The application is based, in part, on the surprising finding that triplex dPCR provides an unbiased method for the detection and quantification of putative rcAAV genomes in rAAV vector manufacturing that is more sensitive, and more widely applicable across AAV serotypes and other manufacturing conditions, than the current gold standard cell-based assays relied upon by rAAV manufacturers.

The methods and kits described herein comprise a multiplex digital polymerase chain reaction (dPCR) amplifying a first junction of an rcAAV genome, a second junction of an rcAAV genome, and a third junction of an rcAAV genome in the rAAV sample, wherein the rcAAV comprises a contiguous 5′ITR, Rep gene, Cap gene, and 3′ITR, and wherein said first junction comprises a nucleotide sequence comprising the 3′ end of the 5′ITR and the 5′ end of the Rep gene; said second junction comprises a nucleotide sequence comprising the 3′ end of the Rep gene and the 5′ end of the Cap gene; said third junction comprises a nucleotide sequence comprising the 3′ end of the Cap gene and the 5′ end of the 3′ITR.

In some aspects, the methods described herein to detect and quantify rcAAV using triplex dPCR of the simultaneous occurrence of ITR-Rep, REP-Cap and Cap-ITR junction fragments comprise an unbiased method to evaluate the presence of rcAAV genomic entities in rAAV vector preparations. Triple positive partitions detected using the methods described herein represent putative rcAAV genomes, whereas particles containing 1 or 2 targeted junctions out of 3 represent partial genomes, and thus are not considered replication competent.

Thus, the present invention in various aspects provides methods and kits for detecting rcAAV in rAAV samples. In another aspect, provided herein are methods for performing quality control on a recombinant adeno-associated virus (rAAV) product.

### II. Definitions

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to #5%, such as less than or equal to +2%, such as less than or equal to +1%, such as less than or equal to +0.5%, such as less than or equal to +0.2%, such as less than or equal to +0.1%, such as less than or equal to +0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the singular forms “a,” “an,” and “the,” refer to both the singular as well as plural, unless the context clearly indicates otherwise. The abbreviation, “e.g.” is derived from the Latin exempli gratia, and is used herein to indicate a non-limiting example. Thus, the abbreviation “e.g.” is synonymous with the term “for example.” As used herein, the term “comprises” means “includes.”

The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith & Waterman Adv. Appl. Math. 2:482, 1981; Needleman & Wunsch J. Mol. Biol. 48:443, 1970; Pearson & Lipman Proc. Natl. Acad. Sci. USA 85:2444, 1988; Higgins & Sharp Gene 73:237-244, 1988; Higgins & Sharp CABIOS 5:151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

The terms, “recombinant AAV” and “rAAV” are used interchangeably herein, and refer to an AAV particle that comprises a nucleotide sequence encoding a protein of interest flanked by inverted terminal repeats (ITRs) that is not capable of replicating under conditions where helper functions are also present.

The terms “replication-competent AAV”, and “rcAAV” are used interchangeably herein, and refer to an AAV particle that comprises a sequence flanked by inverted terminal repeats (ITRs) which is capable of replicating under conditions where helper functions are also present.

A “fluorophore” refers to a molecule that emits light of a certain wavelength after having first absorbed light of a specific, but shorter, wavelength, wherein the emission wavelength is always longer than the absorption wavelength.

The term “quenching agent” refers to a molecule that accepts energy from a fluorophore in the form of light at a particular wavelength and dissipates this energy either in the form of heat (e.g. proximal quenching) or light of a longer wavelength than emitted from the fluorophore (e.g. FRET quenching).

### III. Methods of Detecting Replication Competent Adeno-Associated Virus

In some aspects, provided herein are method of detecting replication competent adeno-associated virus (rcAAV) in a recombinant adeno-associated virus (rAAV) sample, comprising at least one multiplex digital polymerase chain reaction (dPCR) amplifying at least three junctions of a rcAAV genome comprising a first junction of an rcAAV genome, a second junction of an rcAAV genome, and a third junction of an rcAAV genome in the rAAV sample, wherein the rcAAV comprises a contiguous 5′ITR, Rep gene, Cap gene, and 3′ITR, and wherein said first junction comprises a nucleotide sequence comprising the 3′ end of the 5′ITR and the 5′ end of the Rep gene; said second junction comprises a nucleotide sequence comprising the 3′ end of the Rep gene and the 5′ end of the Cap gene; said third junction comprises a nucleotide sequence comprising the 3′ end of the Cap gene and the 5′ end of the 3′ITR. In some aspects, the dPCR method amplifies at least one but no more than three junctions of the rcAAV genome. In other aspects, the dPCR method amplifies at least two but no more than three junctions of the rcAAV genome. In other aspects, the dPCR method amplifies three junctions of the rcAAV genome. In other aspects, the dPCR method amplifies at least one but no more than four junctions of the rcAAV genome. In other aspects, the dPCR method amplifies at least one but no more than five junctions of the rcAAV genome. One of skill in the art could envision adding additional dPCR reactions to amplify additional regions of the rcAAV genome.

In some aspects, provided herein are methods of detecting replication competent adeno-associated virus (rcAAV) in a recombinant adeno-associated virus (rAAV) sample, comprising a multiplex digital polymerase chain reaction (dPCR) comprising; (a) a first dPCR assay comprising the steps of: (i) serially diluting the rAAV sample; (ii) determining the level of each of a first junction of an rcAAV genome, a second junction of an rcAAV genome, and a third junction of an rcAAV genome in the rAAV sample by dPCR; (iii) identifying an optimal dilution for single dPCR quantification of rcAAV in the rAAV sample; and (b) a second dPCR assay comprising the steps of (i) diluting the rAAV sample according to the optimal dilution for single dPCR quantification identified in step (a); (ii) determining the level of each of the first junction of the rcAAV genome, the second junction of the rcAAV genome, and the third junction of the rcAAV genome in the rAAV sample by dPCR; (iii) detecting rcAAV in the rAAV sample when the first junction of the rcAAV genome, the second junction of the rcAAV genome, and the third junction of the rcAAV genome in the rAAV sample are detected within a single partition, wherein the rcAAV comprises a contiguous 5′ITR, Rep gene, Cap gene, and 3′ITR, and wherein said first junction comprises a nucleotide sequence comprising the 3′ end of the 5′ITR and the 5′ end of the Rep gene; said second junction comprises a nucleotide sequence comprising the 3′ end of the Rep gene and the 5′ end of the Cap gene; said third junction comprises a nucleotide sequence comprising the 3′ end of the Cap gene and the 5′ end of the 3′ITR.

In some aspects, the PCR methods described herein utilize partitioning to create multitudes of individual PCR reaction chambers, thereby enabling the precise quantification of nucleic acids by counting the number of positive reactions. In some aspects, the precise quantification of nucleic acids is performed using Poisson statistics to determine the absolute quantity of target nucleic acids without the need for standard curves. PCR methodologies utilizing partitioning may comprise digital PCR (dPCR) or digital droplet PCR (ddPCR). Partitioning in dPCR assays comprises microfluidic chips or nanoplates to create multitudes of individual PCR reaction chambers. Partitioning in dPCR assays is achieved using oil-water emulsions to create multitudes of droplets, each acting as a separate PCR reaction chamber.

In some aspects, the dilution for single dPCR quantification of rcAAV in the rAAV sample is adjusted to reduce the probability of detecting false triple positive partitions. In some aspects, the optimal dilution for single dPCR quantification of rcAAV in the rAAV sample is selected as the largest dilution of the rAAV sample performed in the first dPCR assay satisfying the following criteria; (a) the number of false triple positive partitions comprises less than about 0.1%, less than about 0.09%, less than about 0.08%, less than about 0.07%, less than about 0.06%, less than about 0.05%, less than about 0.04%, less than about 0.03%, less than about 0.02%, or less than about 0.01% of particles, and (b) the number of false double positive partitions comprises less than about 1%, less than about 0.9%, less than about 0.8%, less than about 0.7%, less than about 0.6%, less than about 0.5%, less than about 0.4%, less than about 0.3%, less than about 0.2%, or less than about 0.1% of particles.

In some aspects, the optimal dilution for single dPCR quantification of rcAAV in the rAAV sample is selected as the largest dilution of the rAAV sample performed in the first dPCR assay satisfying the following criteria; (a) the number of triple positive partitions comprises less than about 0.2%, less than about 0.19%, less than about 0.18%, less than about 0.17%, less than about 0.16%, less than about 0.15%, less than about 0.14%, less than about 0.13%, less than about 0.12%, less than about 0.11%, or less than about 0.1% of particles, and (b) the number of double positive partitions comprises less than about 2%, less than about 1.9%, less than about 1.8%, less than about 1.7%, less than about 1.6%, less than about 1.5%, less than about 1.4%, less than about 1.3%, less than about 1.2%, less than about 1.1%, or less than about 1% of particles.

In some aspects, the optimal dilution for single dPCR quantification of rcAAV in the rAAV sample is selected as the largest dilution of the rAAV sample performed in the first dPCR assay satisfying the following criteria; (a) the number of triple positive partitions comprises less than about 0.1% of particles, and (b) the number of double positive partitions comprises less than about 1% of particles.

In some aspects, the rAAV sample is diluted from about 1.25 fold to about 100,000 fold. In some aspects, the rAAV sample is diluted about 1.25 fold, about 1.5 fold, about 1.75 fold, about 2.0 fold, about 2.5 fold, about 3 fold, about 4 fold, about 5 fold, about 10 fold, about 20 fold, about 50 fold, about 100 fold, about 1,000 fold, about 10,000 fold, or about 100,000 fold.

In some aspects the second dPCR assay comprises about 2 to about 200 runs. In some aspects the second dPCR assay comprises about 2, about 4, about 6, about 8, about 10, about 15, about 20, about 30, about 40, about 50, about 100, about 150, or about 200 runs. In some aspects, the minimum number of runs to be employed in the second dPCR assay is determined using the formula:

$$Minimum number of runs = \frac{[ 100 * \frac{\# partitions obtained for a dilution during assay 1}{\begin{matrix}\# positive partitions obtained for a \\ dilution during assay 1\end{matrix}} ]}{maximum \# partitions per well} .$$

In some aspects, the first junction of the rcAAV genome comprises a nucleotide sequence comprising at least a portion of the 3′ end of the 5′ITR and at least a portion of the 5′ end of the Rep gene. In some aspects, the first junction of the rcAAV genome comprises a nucleotide sequence of between about 100 to about 1000 nucleotides. In some aspects, the first junction of the rcAAV genome comprises a nucleotide sequence of about 100, about 200, about 300, about 400, about 500, about 600, about 700, about 800, about 900, or about 1000 nucleotides. In some aspects, the first junction of the rcAAV genome comprises at least about 20, at least about 30, at least about 40, at least about 50, at least about 60, at least about 70, at least about 80, at least about 90, or at least about 100 nucleotides of the 3′ end of the 5′ITR. In some aspects, the first junction of the rcAAV genome comprises at least about 30, at least about 40, at least about 50, at least about 60, at least about 70, at least about 80, at least about 90, or at least about 100 nucleotides of the 5′ end of the Rep gene. In some aspects, the first junction of the rcAAV genome comprises a nucleotide sequence at least 80%, at least 85%, at least 90%, at least 95%, or at least 99% identical to the nucleotide sequence set forth in SEQ ID NO: 13. In some aspects, the first junction of the rcAAV genome comprises a nucleotide sequence at least 80% identical to the nucleotide sequence set forth in SEQ ID NO: 13. In some aspects, the first junction of the rcAAV genome comprises a nucleotide sequence at least 90% identical to the nucleotide sequence set forth in SEQ ID NO:13. In some aspects, the first junction of the rcAAV genome comprises a nucleotide sequence at least 95% identical to the nucleotide sequence set forth in SEQ ID NO:13.

In some aspects, the first junction of the rcAAV genome is amplified using forward/reverse primers capable of hybridizing to at least a portion of the 3′ end of the 5′ITR and at least a portion of the 5′ end of the Rep gene. In some aspects, the first junction of the rcAAV genome is amplified using forward/reverse primers capable of hybridizing to nucleotides 117-137 and 394-412 of the AAV2 genome set forth in SEQ ID NO:1.

In some aspects, the first junction of the rcAAV genome is amplified using a forward primer comprising a nucleotide sequence at least about 80%, at least about 85%, at least about 90%, at least about 95%, or at least about 99% identical to the nucleotide sequence set forth in SEQ ID NO:4, and a reverse primer comprising a nucleotide sequence at least about 80%, at least about 85%, at least about 90%, at least about 95%, or at least about 99% identical to the nucleotide sequence set forth in SEQ ID NO:6. In some aspects, the first junction of the rcAAV genome is amplified using a forward primer comprising a nucleotide sequence at least 80% identical to the nucleotide sequence set forth in SEQ ID NO:4, and a reverse primer comprising a nucleotide sequence at least 80% identical to the nucleotide sequence set forth in SEQ ID NO:6. In some aspects, the first junction of the rcAAV genome is amplified using a forward primer comprising a nucleotide sequence at least 90% identical to the nucleotide sequence set forth in SEQ ID NO:4, and a reverse primer comprising a nucleotide sequence at least 90% identical to the nucleotide sequence set forth in SEQ ID NO:6. In some aspects, the first junction of the rcAAV genome is amplified using a forward primer comprising a nucleotide sequence at least 95% identical to the nucleotide sequence set forth in SEQ ID NO:4, and a reverse primer comprising a nucleotide sequence at least 95% identical to the nucleotide sequence set forth in SEQ ID NO:6. In some aspects, the first junction of the rcAAV genome is amplified using a forward primer comprising the nucleotide sequence set forth in SEQ ID NO:4, and a reverse primer comprising the nucleotide sequence set forth in SEQ ID NO:6.

In some aspects, the first junction of the rcAAV genome is amplified using a forward primer comprising a nucleotide sequence at least about 80%, at least about 85%, at least about 90%, at least about 95%, or at least about 99% identical to the nucleotide sequence set forth in SEQ ID NO:16, and a reverse primer comprising a nucleotide sequence at least about 80%, at least about 85%, at least about 90%, at least about 95%, or at least about 99% identical to the nucleotide sequence set forth in SEQ ID NO: 17. In some aspects, the first junction of a serotype 2, serotype 5, serotype 8, or serotype 9 rcAAV genome is amplified using a forward primer comprising a nucleotide sequence at least about 80%, at least about 85%, at least about 90%, at least about 95%, or at least about 99% identical to the nucleotide sequence set forth in SEQ ID NO: 16, and a reverse primer comprising a nucleotide sequence at least about 80%, at least about 85%, at least about 90%, at least about 95%, or at least about 99% identical to the nucleotide sequence set forth in SEQ ID NO:17. In some aspects, the first junction of the rcAAV genome is amplified using a forward primer comprising a nucleotide sequence at least 80% identical to the nucleotide sequence set forth in SEQ ID NO:16, and a reverse primer comprising a nucleotide sequence at least 80% identical to the nucleotide sequence set forth in SEQ ID NO:17. In some aspects, the first junction of the rcAAV genome is amplified using a forward primer comprising a nucleotide sequence at least 90% identical to the nucleotide sequence set forth in SEQ ID NO:16, and a reverse primer comprising a nucleotide sequence at least 90% identical to the nucleotide sequence set forth in SEQ ID NO:17. In some aspects, the first junction of the rcAAV genome is amplified using a forward primer comprising a nucleotide sequence at least 95% identical to the nucleotide sequence set forth in SEQ ID NO:16, and a reverse primer comprising a nucleotide sequence at least 95% identical to the nucleotide sequence set forth in SEQ ID NO: 17. In some aspects, the first junction of the rcAAV genome is amplified using a forward primer comprising the nucleotide sequence set forth in SEQ ID NO: 16, and a reverse primer comprising the nucleotide sequence set forth in SEQ ID NO:17.

In some aspects, the second junction of the rcAAV genome comprises a nucleotide sequence comprising at least a portion of the 3′ end of the Rep gene and at least a portion of the 5′ end of the Cap gene. In some aspects, the second junction of the rcAAV genome comprises a nucleotide sequence of between about 100 to about 1000 nucleotides. In some aspects, the second junction of the rcAAV genome comprises a nucleotide sequence of about 100, about 200, about 300, about 400, about 500, about 600, about 700, about 800, about 900, or about 1000 nucleotides. In some aspects, the second junction of the rcAAV genome comprises at least about 20, at least about 30, at least about 40, at least about 50, at least about 60, at least about 70, at least about 80, at least about 90, or at least about 100 nucleotides of the 3′ end of the Rep gene. In some aspects, the second junction of the rcAAV genome comprises at least about 20, at least about 30, at least about 40, at least about 50, at least about 60, at least about 70, at least about 80, at least about 90, or at least about 100 nucleotides of the 5′ end of the Cap gene. In some aspects, the second junction of the rcAAV genome comprises a nucleotide sequence at least 80%, at least 85%, at least 90%, at least 95%, or at least 99% identical to the nucleotide sequence set forth in SEQ ID NO:14. In some aspects, the second junction of the rcAAV genome comprises a nucleotide sequence at least 80% identical to the nucleotide sequence set forth in SEQ ID NO:14.

In some aspects, the second junction of the rcAAV genome is amplified using forward/reverse primers capable of hybridizing to at least a portion of the 3′ end of the Rep gene and at least a portion of the 5′ end of the Cap gene. In some aspects, the second junction of the rcAAV genome is amplified using forward/reverse primers capable of hybridizing to nucleotides 2156-2178 and 2156-2277 of the AAV2 genome set forth in SEQ ID NO:1.

In some aspects, the second junction of the rcAAV genome is amplified using a forward primer comprising a nucleotide sequence at least about 80%, at least about 85%, at least about 90%, at least about 95%, or at least about 99% identical to the nucleotide sequence set forth in SEQ ID NO:8, and a reverse primer comprising a nucleotide sequence at least about 80%, at least about 85%, at least about 90%, at least about 95%, or at least about 99% identical to the nucleotide sequence set forth in SEQ ID NO:9. In some aspects, the second junction of a serotype 2 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:8, and a reverse primer comprising a nucleotide sequence at least about 80%, at least about 85%, at least about 90%, at least about 95%, or at least about 99% identical to the nucleotide sequence set forth in SEQ ID NO:9. In some aspects, the second junction of the rcAAV genome is amplified using a forward primer comprising a nucleotide sequence at least 80% identical to the nucleotide sequence set forth in SEQ ID NO:8, and a reverse primer comprising a nucleotide sequence at least 80% identical to the nucleotide sequence set forth in SEQ ID NO:9. In some aspects, the second junction of the rcAAV genome is amplified using a forward primer comprising a nucleotide sequence at least 90% identical to the nucleotide sequence set forth in SEQ ID NO:8, and a reverse primer comprising a nucleotide sequence at least 90% identical to the nucleotide sequence set forth in SEQ ID NO:9. In some aspects, the second junction of the rcAAV genome is amplified using a forward primer comprising a nucleotide sequence at least 95% identical to the nucleotide sequence set forth in SEQ ID NO:8, and a reverse primer comprising a nucleotide sequence at least 95% identical to the nucleotide sequence set forth in SEQ ID NO:9. In some aspects, the second junction of the rcAAV genome is amplified using a forward primer comprising the nucleotide sequence set forth in SEQ ID NO:8, and a reverse primer comprising the nucleotide sequence set forth in SEQ ID NO:9.

In some aspects, the second junction of a serotype 2 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:22, and a reverse primer comprising a nucleotide sequence at least about 80%, at least about 85%, at least about 90%, at least about 95%, or at least about 99% identical to the nucleotide sequence set forth in SEQ ID NO:23. In some aspects, the second junction of the rcAAV genome is amplified using a forward primer comprising a nucleotide sequence at least 80% identical to the nucleotide sequence set forth in SEQ ID NO:22, and a reverse primer comprising a nucleotide sequence at least 80% identical to the nucleotide sequence set forth in SEQ ID NO:23. In some aspects, the second junction of the rcAAV genome is amplified using a forward primer comprising a nucleotide sequence at least 90% identical to the nucleotide sequence set forth in SEQ ID NO:22, and a reverse primer comprising a nucleotide sequence at least 90% identical to the nucleotide sequence set forth in SEQ ID NO:23. In some aspects, the second junction of the rcAAV genome is amplified using a forward primer comprising a nucleotide sequence at least 95% identical to the nucleotide sequence set forth in SEQ ID NO:22, and a reverse primer comprising a nucleotide sequence at least 95% identical to the nucleotide sequence set forth in SEQ ID NO:23. In some aspects, the second junction of the rcAAV genome is amplified using a forward primer comprising the nucleotide sequence set forth in SEQ ID NO:22, and a reverse primer comprising the nucleotide sequence set forth in SEQ ID NO:23.

In some aspects, the second junction of a serotype 5 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:28, and a reverse primer comprising a nucleotide sequence at least about 80%, at least about 85%, at least about 90%, at least about 95%, or at least about 99% identical to the nucleotide sequence set forth in SEQ ID NO:29. In some aspects, the second junction of the rcAAV genome is amplified using a forward primer comprising a nucleotide sequence at least 80% identical to the nucleotide sequence set forth in SEQ ID NO:28, and a reverse primer comprising a nucleotide sequence at least 80% identical to the nucleotide sequence set forth in SEQ ID NO:29. In some aspects, the second junction of the rcAAV genome is amplified using a forward primer comprising a nucleotide sequence at least 90% identical to the nucleotide sequence set forth in SEQ ID NO:28, and a reverse primer comprising a nucleotide sequence at least 90% identical to the nucleotide sequence set forth in SEQ ID NO:29. In some aspects, the second junction of the rcAAV genome is amplified using a forward primer comprising a nucleotide sequence at least 95% identical to the nucleotide sequence set forth in SEQ ID NO:28, and a reverse primer comprising a nucleotide sequence at least 95% identical to the nucleotide sequence set forth in SEQ ID NO:29. In some aspects, the second junction of the rcAAV genome is amplified using a forward primer comprising the nucleotide sequence set forth in SEQ ID NO:28, and a reverse primer comprising the nucleotide sequence set forth in SEQ ID NO:29.

In some aspects, the second junction of a serotype 8 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:34, and a reverse primer comprising a nucleotide sequence at least about 80%, at least about 85%, at least about 90%, at least about 95%, or at least about 99% identical to the nucleotide sequence set forth in SEQ ID NO:35. In some aspects, the second junction of the rcAAV genome is amplified using a forward primer comprising a nucleotide sequence at least 80% identical to the nucleotide sequence set forth in SEQ ID NO:34, and a reverse primer comprising a nucleotide sequence at least 80% identical to the nucleotide sequence set forth in SEQ ID NO:35. In some aspects, the second junction of the rcAAV genome is amplified using a forward primer comprising a nucleotide sequence at least 90% identical to the nucleotide sequence set forth in SEQ ID NO:34, and a reverse primer comprising a nucleotide sequence at least 90% identical to the nucleotide sequence set forth in SEQ ID NO:35. In some aspects, the second junction of the rcAAV genome is amplified using a forward primer comprising a nucleotide sequence at least 95% identical to the nucleotide sequence set forth in SEQ ID NO:34, and a reverse primer comprising a nucleotide sequence at least 95% identical to the nucleotide sequence set forth in SEQ ID NO:35. In some aspects, the second junction of the rcAAV genome is amplified using a forward primer comprising the nucleotide sequence set forth in SEQ ID NO:34, and a reverse primer comprising the nucleotide sequence set forth in SEQ ID NO:35.

In some aspects, the second junction of a serotype 9 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:40, and a reverse primer comprising a nucleotide sequence at least about 80%, at least about 85%, at least about 90%, at least about 95%, or at least about 99% identical to the nucleotide sequence set forth in SEQ ID NO:41. In some aspects, the second junction of the rcAAV genome is amplified using a forward primer comprising a nucleotide sequence at least 80% identical to the nucleotide sequence set forth in SEQ ID NO:40, and a reverse primer comprising a nucleotide sequence at least 80% identical to the nucleotide sequence set forth in SEQ ID NO:41. In some aspects, the second junction of the rcAAV genome is amplified using a forward primer comprising a nucleotide sequence at least 90% identical to the nucleotide sequence set forth in SEQ ID NO:40, and a reverse primer comprising a nucleotide sequence at least 90% identical to the nucleotide sequence set forth in SEQ ID NO:41. In some aspects, the second junction of the rcAAV genome is amplified using a forward primer comprising a nucleotide sequence at least 95% identical to the nucleotide sequence set forth in SEQ ID NO:40, and a reverse primer comprising a nucleotide sequence at least 95% identical to the nucleotide sequence set forth in SEQ ID NO:41. In some aspects, the second junction of the rcAAV genome is amplified using a forward primer comprising the nucleotide sequence set forth in SEQ ID NO:40, and a reverse primer comprising the nucleotide sequence set forth in SEQ ID NO:41.

In some aspects, the third junction of the rcAAV genome comprises a nucleotide sequence comprising at least a portion of the 3′ end of the Cap gene and at least a portion of the 5′ end of the 3′ITR. In some aspects, the third junction of the rcAAV comprises a nucleotide sequence of between about 100 to about 1000 nucleotides. In some aspects, the third junction of the rcAAV genome comprises a nucleotide sequence of about 100, about 200, about 300, about 400, about 500, about 600, about 700, about 800, about 900, or about 1000 nucleotides. In some aspects, the third junction of the rcAAV genome comprises at least about 20, at least about 30, at least about 40, at least about 50, at least about 60, at least about 70, at least about 80, at least about 90, or at least about 100 nucleotides of the 3′ end of the Cap gene. In some aspects, the third junction of the rcAAV genome comprises at least about 20, at least about 30, at least about 40, at least about 50, at least about 60, at least about 70, at least about 80, at least about 90, or at least about 100 nucleotides of the 5′ end of the 3′ITR. In some aspects, the third junction of the rcAAV genome comprises a nucleotide sequence at least 80%, at least 85%, at least 90%, at least 95%, or at least 99% identical to the nucleotide sequence set forth in SEQ ID NO:15.

In some aspects, the third junction of the rcAAV genome is amplified using forward/reverse primers capable of hybridizing to at least a portion of the 3′ end of the Cap gene and at least a portion of the 5′ end of the 3′ITR. In some aspects, the third junction of the rcAAV genome is amplified using forward/reverse primers capable of hybridizing to nucleotides 4342-4363 and 4543-4563 of the AAV2 genome set forth in SEQ ID NO:1.

In some aspects, the third junction of the rcAAV genome is amplified using a forward primer comprising a nucleotide sequence at least about 80%, at least about 85%, at least about 90%, at least about 95%, or at least about 99% identical to the nucleotide sequence set forth in SEQ ID NO:7, and a reverse primer comprising a nucleotide sequence at least about 80%, at least about 85%, at least about 90%, at least about 95%, or at least about 99% identical to the nucleotide sequence set forth in SEQ ID NO:5. In some aspects, the third junction of a serotype 2 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:7, and a reverse primer comprising a nucleotide sequence at least about 80%, at least about 85%, at least about 90%, at least about 95%, or at least about 99% identical to the nucleotide sequence set forth in SEQ ID NO:5. In some aspects, the third junction of the rcAAV genome is amplified using a forward primer comprising a nucleotide sequence at least 80% identical to the nucleotide sequence set forth in SEQ ID NO:7, and a reverse primer comprising a nucleotide sequence at least 80% identical to the nucleotide sequence set forth in SEQ ID NO:5. In some aspects, the third junction of the rcAAV genome is amplified using a forward primer comprising a nucleotide sequence at least 90% identical to the nucleotide sequence set forth in SEQ ID NO:7, and a reverse primer comprising a nucleotide sequence at least 90% identical to the nucleotide sequence set forth in SEQ ID NO:5. In some aspects, the third junction of the rcAAV genome is amplified using a forward primer comprising a nucleotide sequence at least 95% identical to the nucleotide sequence set forth in SEQ ID NO:7, and a reverse primer comprising a nucleotide sequence at least 95% identical to the nucleotide sequence set forth in SEQ ID NO:5. In some aspects, the third junction of the rcAAV genome is amplified using a forward primer comprising the nucleotide sequence set forth in SEQ ID NO:7, and a reverse primer comprising the nucleotide sequence set forth in SEQ ID NO:5.

In some aspects, the third junction of a serotype 2 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:19, and a reverse primer comprising a nucleotide sequence at least about 80%, at least about 85%, at least about 90%, at least about 95%, or at least about 99% identical to the nucleotide sequence set forth in SEQ ID NO:20. In some aspects, the third junction of the rcAAV genome is amplified using a forward primer comprising a nucleotide sequence at least 80% identical to the nucleotide sequence set forth in SEQ ID NO:19, and a reverse primer comprising a nucleotide sequence at least 80% identical to the nucleotide sequence set forth in SEQ ID NO:20. In some aspects, the third junction of the rcAAV genome is amplified using a forward primer comprising a nucleotide sequence at least 90% identical to the nucleotide sequence set forth in SEQ ID NO:19, and a reverse primer comprising a nucleotide sequence at least 90% identical to the nucleotide sequence set forth in SEQ ID NO:20. In some aspects, the third junction of the rcAAV genome is amplified using a forward primer comprising a nucleotide sequence at least 95% identical to the nucleotide sequence set forth in SEQ ID NO:19, and a reverse primer comprising a nucleotide sequence at least 95% identical to the nucleotide sequence set forth in SEQ ID NO:20. In some aspects, the third junction of the rcAAV genome is amplified using a forward primer comprising the nucleotide sequence set forth in SEQ ID NO: 19, and a reverse primer comprising the nucleotide sequence set forth in SEQ ID NO:20.

In some aspects, the third junction of a serotype 5 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:25, and a reverse primer comprising a nucleotide sequence at least about 80%, at least about 85%, at least about 90%, at least about 95%, or at least about 99% identical to the nucleotide sequence set forth in SEQ ID NO:26. In some aspects, the third junction of the rcAAV genome is amplified using a forward primer comprising a nucleotide sequence at least 80% identical to the nucleotide sequence set forth in SEQ ID NO:25, and a reverse primer comprising a nucleotide sequence at least 80% identical to the nucleotide sequence set forth in SEQ ID NO:26. In some aspects, the third junction of the rcAAV genome is amplified using a forward primer comprising a nucleotide sequence at least 90% identical to the nucleotide sequence set forth in SEQ ID NO:25, and a reverse primer comprising a nucleotide sequence at least 90% identical to the nucleotide sequence set forth in SEQ ID NO:26. In some aspects, the third junction of the rcAAV genome is amplified using a forward primer comprising a nucleotide sequence at least 95% identical to the nucleotide sequence set forth in SEQ ID NO:25, and a reverse primer comprising a nucleotide sequence at least 95% identical to the nucleotide sequence set forth in SEQ ID NO:26. In some aspects, the third junction of the rcAAV genome is amplified using a forward primer comprising the nucleotide sequence set forth in SEQ ID NO:25, and a reverse primer comprising the nucleotide sequence set forth in SEQ ID NO:26.

In some aspects, the third junction of a serotype 8 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:31, and a reverse primer comprising a nucleotide sequence at least about 80%, at least about 85%, at least about 90%, at least about 95%, or at least about 99% identical to the nucleotide sequence set forth in SEQ ID NO:32. In some aspects, the third junction of the rcAAV genome is amplified using a forward primer comprising a nucleotide sequence at least 80% identical to the nucleotide sequence set forth in SEQ ID NO:31, and a reverse primer comprising a nucleotide sequence at least 80% identical to the nucleotide sequence set forth in SEQ ID NO:32. In some aspects, the third junction of the rcAAV genome is amplified using a forward primer comprising a nucleotide sequence at least 90% identical to the nucleotide sequence set forth in SEQ ID NO:31, and a reverse primer comprising a nucleotide sequence at least 90% identical to the nucleotide sequence set forth in SEQ ID NO:32. In some aspects, the third junction of the rcAAV genome is amplified using a forward primer comprising a nucleotide sequence at least 95% identical to the nucleotide sequence set forth in SEQ ID NO:31, and a reverse primer comprising a nucleotide sequence at least 95% identical to the nucleotide sequence set forth in SEQ ID NO:32. In some aspects, the third junction of the rcAAV genome is amplified using a forward primer comprising the nucleotide sequence set forth in SEQ ID NO:31, and a reverse primer comprising the nucleotide sequence set forth in SEQ ID NO:32.

In some aspects, the third junction of a serotype 9 rcAAV genome is amplified using a forward primer comprising a nucleotide sequence at least about 80%, at least about 85%, at least about 90%, at least about 95%, or at least about 99% identical to the nucleotide sequence set forth in SEQ ID NO:37, and a reverse primer comprising a nucleotide sequence at least about 80%, at least about 85%, at least about 90%, at least about 95%, or at least about 99% identical to the nucleotide sequence set forth in SEQ ID NO:38. In some aspects, the third junction of the rcAAV genome is amplified using a forward primer comprising a nucleotide sequence at least 80% identical to the nucleotide sequence set forth in SEQ ID NO:37, and a reverse primer comprising a nucleotide sequence at least 80% identical to the nucleotide sequence set forth in SEQ ID NO:38. In some aspects, the third junction of the rcAAV genome is amplified using a forward primer comprising a nucleotide sequence at least 90% identical to the nucleotide sequence set forth in SEQ ID NO:37, and a reverse primer comprising a nucleotide sequence at least 90% identical to the nucleotide sequence set forth in SEQ ID NO:38. In some aspects, the third junction of the rcAAV genome is amplified using a forward primer comprising a nucleotide sequence at least 95% identical to the nucleotide sequence set forth in SEQ ID NO:37, and a reverse primer comprising a nucleotide sequence at least 95% identical to the nucleotide sequence set forth in SEQ ID NO:38. In some aspects, the third junction of the rcAAV genome is amplified using a forward primer comprising the nucleotide sequence set forth in SEQ ID NO:37, and a reverse primer comprising the nucleotide sequence set forth in SEQ ID NO:38.

In some aspects, the provided methods involve analyzing, e.g., detecting or quantifying, one or more sequences present in a dPCR amplification product. In some aspects, analyzing (e.g., detecting or quantifying) the one or more sequences in the dPCR amplification product comprises detecting a complex comprising the one or more dPCR amplification products and one or more fluorescently labeled hydrolysis probes. In some aspects, the first junction of the rcAAV genome, the second junction of the rcAAV genome, and the third junction of the rcAAV genome are detected in the first dPCR assay and the second dPCR assay using fluorescently labeled hydrolysis probes. In some aspects, determining the level of each of the first junction of the rcAAV genome, the second junction of the rcAAV genome, and the third junction of the rcAAV genome comprises determining the quantity of the first junction of the rcAAV genome, the second junction of the rcAAV genome, and the third junction of the rcAAV genome in the rAAV sample. In some aspects, determining the level of each of the first junction of the rcAAV genome, the second junction of the rcAAV genome, and the third junction of the rcAAV genome comprises determining the amount of the first junction of the rcAAV genome, the second junction of the rcAAV genome, and the third junction of the rcAAV genome in the rAAV sample.

A fluorescently labeled hydrolysis probe herein comprises a detectable label (i.e., a fluorophore) at one end of the hydrolysis probe, a quencher dye at the other end of the hydrolysis probe, and a nucleotide sequence complementary to a nucleotide sequence in a first junction of an rcAAV genome, a second junction of an rcAAV genome, or a third junction of an rcAAV genome between the detectable label and quencher dye. During the annealing phase of a dPCR cycle, the fluorescently labeled hydrolysis probe binds to its complementary target sequence on the dPCR amplification product. As the DNA polymerase extends the primer and synthesizes the new DNA strand, it encounters the bound probe. The 5′ to 3′ exonuclease activity of the dPCR reaction's DNA polymerase cleaves the probe, separating the reporter dye from the quencher. Once the reporter dye is separated from the quencher, it emits fluorescence. This fluorescence signal increases proportionally with the amount of target DNA present in the reaction.

In some aspects, the fluorescently labeled hydrolysis probes are individually between about 15 and about 25 nucleotides in length, between about 15 and about 30 nucleotides in length, between about 15 and about 40 nucleotides in length, or between about 20 and about 40 nucleotides in length. In some other aspects, the nucleotide sequence complementary to the first junction of the rcAAV genome, the second junction of the rcAAV genome, or the third junction of the rcAAV genome are each between about 15 and about 25 nucleotides in length, between about 15 and about 30 nucleotides in length, between about 15 and about 40 nucleotides in length, or between about 20 and about 40 nucleotides in length.

In some aspects, the detecting comprises imaging the dPCR partitions comprising the dPCR amplification products. In some such aspects, the detecting comprises determining a signal, e.g., a fluorescent signal. In some aspects, fluorescence microscopy is used for detection and imaging of the fluorescently labeled hydrolysis probes. In some aspects, the fluorescently labeled hydrolysis probe comprises a fluorophore having an excitation peak between 480 nm and 500 nm. In some aspects, the fluorescently labeled hydrolysis probe comprises a fluorophore having an excitation peak between 520 nm and 540 nm. In some aspects, the fluorescently labeled hydrolysis probe comprises a fluorophore having an excitation peak between 590 nm and 600 nm. In some aspects, the fluorescently labeled hydrolysis probe comprises a fluorophore having an excitation peak between 640 nm and 660 nm.

In some aspects, the fluorescently labeled hydrolysis probes comprise a fluorescent label selected from the group consisting of 6-Carboxyfluorescein (FAM), ATTO 488, Tetrachlorofluorescein (TET), ATTO 532, Hexachlorofluorescein (HEX), JOE, TYE 563, Cy 3, ATTO 550, Carboxytetramethyl-rhodamine (TAMRA), ATTO 565, ROX, ATTO Rho 101, TEX 615, and Texas Red-X.

In some aspects, the one or more fluorescently labeled hydrolysis probes comprise a quenching agent. The one or more fluorescently labeled hydrolysis probes may comprise a quenching agent at either the 5′ or 3′ end. Preferably, the quenching agent is present at the opposite end of the hydrolysis probe to the detectable label. For example, if the detectable label is present at the 5′ end of the probe, the quenching agent is preferably present at the 3′ end of the probe. In some aspects, the quenching agent is selected from the group consisting of Iowa Black RQ, Black Hole Quencher-2, ZEN-Iowa Black FQ, and Black Hole Quencher-1.

In some aspects, the first junction of the rcAAV genome is detected using a fluorescently labeled hydrolysis probe capable of hybridizing to at least a portion of a sequence corresponding to nucleotides 357-383 of the AAV2 genome set forth in SEQ ID NO:1. In some aspects, the first junction of the rcAAV genome is detected using a fluorescently labeled hydrolysis probe comprising a nucleotide sequence at least 80% identical to the nucleotide sequence set forth in SEQ ID NO:10. In some aspects, the first junction of a serotype 2 rcAAV genome is detected using a fluorescently labeled hydrolysis probe comprising a nucleotide sequence at least 80% identical to the nucleotide sequence set forth in SEQ ID NO:10. In some aspects, the first junction of the rcAAV genome is detected using a fluorescently labeled hydrolysis probe comprising a nucleotide sequence at least 90% identical to the nucleotide sequence set forth in SEQ ID NO: 10. In some aspects, the first junction of the rcAAV genome is detected using a fluorescently labeled hydrolysis probe comprising a nucleotide sequence at least 95% identical to the nucleotide sequence set forth in SEQ ID NO:10. In some aspects, the first junction of the rcAAV genome is detected using a fluorescently labeled hydrolysis probe comprising a nucleotide sequence at least 99% identical to the nucleotide sequence set forth in SEQ ID NO:10. In some aspects, the first junction of the rcAAV genome is detected using a fluorescently labeled hydrolysis probe comprising the nucleotide sequence set forth in SEQ ID NO:10. In some aspects, the first junction of a serotype 2 rcAAV genome is detected using a fluorescently labeled hydrolysis probe comprising a nucleotide sequence at least 80% identical to the nucleotide sequence set forth in SEQ ID NO:18. In some aspects, the first junction of the rcAAV genome is detected using a fluorescently labeled hydrolysis probe comprising a nucleotide sequence at least 90% identical to the nucleotide sequence set forth in SEQ ID NO: 18. In some aspects, the first junction of the rcAAV genome is detected using a fluorescently labeled hydrolysis probe comprising a nucleotide sequence at least 95% identical to the nucleotide sequence set forth in SEQ ID NO: 18. In some aspects, the first junction of the rcAAV genome is detected using a fluorescently labeled hydrolysis probe comprising a nucleotide sequence at least 99% identical to the nucleotide sequence set forth in SEQ ID NO:18. In some aspects, the first junction of the rcAAV genome is detected using a fluorescently labeled hydrolysis probe comprising the nucleotide sequence set forth in SEQ ID NO:18.

In some aspects, the second junction of the rcAAV genome is detected using a fluorescently labeled hydrolysis probe capable of hybridizing to at least a portion of a sequence corresponding to nucleotides 2203-2226 of the AAV2 genome set forth in SEQ ID NO:1. In some aspects, the second junction of the rcAAV genome is detected using a fluorescently labeled hydrolysis probe comprising a nucleotide sequence at least 80% identical to the nucleotide sequence set forth in SEQ ID NO:12. In some aspects, the second junction of a serotype 2 rcAAV genome is detected using a fluorescently labeled hydrolysis probe comprising a nucleotide sequence at least 80% identical to the nucleotide sequence set forth in SEQ ID NO:12. In some aspects, the second junction of the rcAAV genome is detected using a fluorescently labeled hydrolysis probe comprising a nucleotide sequence at least 90% identical to the nucleotide sequence set forth in SEQ ID NO:12. In some aspects, the second junction of the rcAAV genome is detected using a fluorescently labeled hydrolysis probe comprising a nucleotide sequence at least 95% identical to the nucleotide sequence set forth in SEQ ID NO:12. In some aspects, the second junction of the rcAAV genome is detected using a fluorescently labeled hydrolysis probe comprising a nucleotide sequence at least 99% identical to the nucleotide sequence set forth in SEQ ID NO:12. In some aspects, the second junction of the rcAAV genome is detected using a fluorescently labeled hydrolysis probe comprising the nucleotide sequence set forth in SEQ ID NO:12.

In some aspects, the second junction of a serotype 2 rcAAV genome is detected using a fluorescently labeled hydrolysis probe comprising a nucleotide sequence at least 80% identical to the nucleotide sequence set forth in SEQ ID NO:24. In some aspects, the second junction of the rcAAV genome is detected using a fluorescently labeled hydrolysis probe comprising a nucleotide sequence at least 90% identical to the nucleotide sequence set forth in SEQ ID NO:24. In some aspects, the second junction of the rcAAV genome is detected using a fluorescently labeled hydrolysis probe comprising a nucleotide sequence at least 95% identical to the nucleotide sequence set forth in SEQ ID NO:24. In some aspects, the second junction of the rcAAV genome is detected using a fluorescently labeled hydrolysis probe comprising a nucleotide sequence at least 99% identical to the nucleotide sequence set forth in SEQ ID NO:24. In some aspects, the second junction of the rcAAV genome is detected using a fluorescently labeled hydrolysis probe comprising the nucleotide sequence set forth in SEQ ID NO:24.

In some aspects, the second junction of a serotype 5 rcAAV genome is detected using a fluorescently labeled hydrolysis probe comprising a nucleotide sequence at least 80% identical to the nucleotide sequence set forth in SEQ ID NO:30. In some aspects, the second junction of the rcAAV genome is detected using a fluorescently labeled hydrolysis probe comprising a nucleotide sequence at least 90% identical to the nucleotide sequence set forth in SEQ ID NO:30. In some aspects, the second junction of the rcAAV genome is detected using a fluorescently labeled hydrolysis probe comprising a nucleotide sequence at least 95% identical to the nucleotide sequence set forth in SEQ ID NO:30. In some aspects, the second junction of the rcAAV genome is detected using a fluorescently labeled hydrolysis probe comprising a nucleotide sequence at least 99% identical to the nucleotide sequence set forth in SEQ ID NO:30. In some aspects, the second junction of the rcAAV genome is detected using a fluorescently labeled hydrolysis probe comprising the nucleotide sequence set forth in SEQ ID NO:30.

In some aspects, the second junction of a serotype 8 rcAAV genome is detected using a fluorescently labeled hydrolysis probe comprising a nucleotide sequence at least 80% identical to the nucleotide sequence set forth in SEQ ID NO:36. In some aspects, the second junction of the rcAAV genome is detected using a fluorescently labeled hydrolysis probe comprising a nucleotide sequence at least 90% identical to the nucleotide sequence set forth in SEQ ID NO:36. In some aspects, the second junction of the rcAAV genome is detected using a fluorescently labeled hydrolysis probe comprising a nucleotide sequence at least 95% identical to the nucleotide sequence set forth in SEQ ID NO:36. In some aspects, the second junction of the rcAAV genome is detected using a fluorescently labeled hydrolysis probe comprising a nucleotide sequence at least 99% identical to the nucleotide sequence set forth in SEQ ID NO:36. In some aspects, the second junction of the rcAAV genome is detected using a fluorescently labeled hydrolysis probe comprising the nucleotide sequence set forth in SEQ ID NO:36.

In some aspects, the second junction of a serotype 9 rcAAV genome is detected using a fluorescently labeled hydrolysis probe comprising a nucleotide sequence at least 80% identical to the nucleotide sequence set forth in SEQ ID NO:42. In some aspects, the second junction of the rcAAV genome is detected using a fluorescently labeled hydrolysis probe comprising a nucleotide sequence at least 90% identical to the nucleotide sequence set forth in SEQ ID NO:42. In some aspects, the second junction of the rcAAV genome is detected using a fluorescently labeled hydrolysis probe comprising a nucleotide sequence at least 95% identical to the nucleotide sequence set forth in SEQ ID NO:42. In some aspects, the second junction of the rcAAV genome is detected using a fluorescently labeled hydrolysis probe comprising a nucleotide sequence at least 99% identical to the nucleotide sequence set forth in SEQ ID NO:42. In some aspects, the second junction of the rcAAV genome is detected using a fluorescently labeled hydrolysis probe comprising the nucleotide sequence set forth in SEQ ID NO:42.

In some aspects, the third junction of the rcAAV genome is detected using a fluorescently labeled hydrolysis probe capable of hybridizing to at least a portion of a sequence corresponding to nucleotides 4375-4398 of the AAV2 genome set forth in SEQ ID NO:1. In some aspects, the third junction of the rcAAV genome is detected using a fluorescently labeled hydrolysis probe comprising a nucleotide sequence at least 80% identical to the nucleotide sequence set forth in SEQ ID NO:11. In some aspects, the third junction of a serotype 2 rcAAV genome is detected using a fluorescently labeled hydrolysis probe comprising a nucleotide sequence at least 80% identical to the nucleotide sequence set forth in SEQ ID NO:11. In some aspects, the third junction of the rcAAV genome is detected using a fluorescently labeled hydrolysis probe comprising a nucleotide sequence at least 90% identical to the nucleotide sequence set forth in SEQ ID NO: 11. In some aspects, the third junction of the rcAAV genome is detected using a fluorescently labeled hydrolysis probe comprising a nucleotide sequence at least 95% identical to the nucleotide sequence set forth in SEQ ID NO: 11. In some aspects, the third junction of the rcAAV genome is detected using a fluorescently labeled hydrolysis probe comprising a nucleotide sequence at least 99% identical to the nucleotide sequence set forth in SEQ ID NO: 11. In some aspects, the third junction of the rcAAV genome is detected using a fluorescently labeled hydrolysis probe comprising the nucleotide sequence set forth in SEQ ID NO:11.

In some aspects, the third junction of a serotype 2 rcAAV genome is detected using a fluorescently labeled hydrolysis probe comprising a nucleotide sequence at least 80% identical to the nucleotide sequence set forth in SEQ ID NO:21. In some aspects, the third junction of the rcAAV genome is detected using a fluorescently labeled hydrolysis probe comprising a nucleotide sequence at least 90% identical to the nucleotide sequence set forth in SEQ ID NO:21. In some aspects, the third junction of the rcAAV genome is detected using a fluorescently labeled hydrolysis probe comprising a nucleotide sequence at least 95% identical to the nucleotide sequence set forth in SEQ ID NO:21. In some aspects, the third junction of the rcAAV genome is detected using a fluorescently labeled hydrolysis probe comprising a nucleotide sequence at least 99% identical to the nucleotide sequence set forth in SEQ ID NO:21. In some aspects, the third junction of the rcAAV genome is detected using a fluorescently labeled hydrolysis probe comprising the nucleotide sequence set forth in SEQ ID NO:21.

In some aspects, the third junction of a serotype 5 rcAAV genome is detected using a fluorescently labeled hydrolysis probe comprising a nucleotide sequence at least 80% identical to the nucleotide sequence set forth in SEQ ID NO:27. In some aspects, the third junction of the rcAAV genome is detected using a fluorescently labeled hydrolysis probe comprising a nucleotide sequence at least 90% identical to the nucleotide sequence set forth in SEQ ID NO:27. In some aspects, the third junction of the rcAAV genome is detected using a fluorescently labeled hydrolysis probe comprising a nucleotide sequence at least 95% identical to the nucleotide sequence set forth in SEQ ID NO:27. In some aspects, the third junction of the rcAAV genome is detected using a fluorescently labeled hydrolysis probe comprising a nucleotide sequence at least 99% identical to the nucleotide sequence set forth in SEQ ID NO:27. In some aspects, the third junction of the rcAAV genome is detected using a fluorescently labeled hydrolysis probe comprising the nucleotide sequence set forth in SEQ ID NO:27.

In some aspects, the third junction of a serotype 8 rcAAV genome is detected using a fluorescently labeled hydrolysis probe comprising a nucleotide sequence at least 80% identical to the nucleotide sequence set forth in SEQ ID NO:33. In some aspects, the third junction of the rcAAV genome is detected using a fluorescently labeled hydrolysis probe comprising a nucleotide sequence at least 90% identical to the nucleotide sequence set forth in SEQ ID NO:33. In some aspects, the third junction of the rcAAV genome is detected using a fluorescently labeled hydrolysis probe comprising a nucleotide sequence at least 95% identical to the nucleotide sequence set forth in SEQ ID NO:33. In some aspects, the third junction of the rcAAV genome is detected using a fluorescently labeled hydrolysis probe comprising a nucleotide sequence at least 99% identical to the nucleotide sequence set forth in SEQ ID NO:33. In some aspects, the third junction of the rcAAV genome is detected using a fluorescently labeled hydrolysis probe comprising the nucleotide sequence set forth in SEQ ID NO:33.

In some aspects, the third junction of a serotype 9 rcAAV genome is detected using a fluorescently labeled hydrolysis probe comprising a nucleotide sequence at least 80% identical to the nucleotide sequence set forth in SEQ ID NO:39. In some aspects, the third junction of the rcAAV genome is detected using a fluorescently labeled hydrolysis probe comprising a nucleotide sequence at least 90% identical to the nucleotide sequence set forth in SEQ ID NO:39. In some aspects, the third junction of the rcAAV genome is detected using a fluorescently labeled hydrolysis probe comprising a nucleotide sequence at least 95% identical to the nucleotide sequence set forth in SEQ ID NO:39. In some aspects, the third junction of the rcAAV genome is detected using a fluorescently labeled hydrolysis probe comprising a nucleotide sequence at least 99% identical to the nucleotide sequence set forth in SEQ ID NO:39. In some aspects, the third junction of the rcAAV genome is detected using a fluorescently labeled hydrolysis probe comprising the nucleotide sequence set forth in SEQ ID NO:39.

### IV. Methods of Producing Recombinant Adeno-associated Virus

In some aspects, disclosed herein is a method of producing a recombinant adeno-associated virus (rAAV), the method comprising a) recombinantly producing the AAV; b) collecting the rAAV from step (a) media comprising an AAV host cell; and c) quantifying the amount of rcAAV contaminants in the sample using the method of detecting rcAAV disclosed herein; and d) discarding the produced rAAV product when rcAAV is detectibly present or detectable above a threshold, or not-discarding the produced AAV when rcAAV is not detectibly present or detectable below a threshold.

Methods for recombinantly producing AAV samples have been described in, e.g., U.S. Pat. No. 6,593,123, which is hereby incorporated herein by reference in its entirety.

In some aspects, recombinantly producing an AAV comprises delivering one or more vectors to host cells comprising a transgene expression construct, rep sequences, cap sequences, and helper functions required for producing rAAV products.

In some aspects, the transgene expression construct comprises a transgene and its regulatory sequences, and 5′ and 3′ AAV inverted terminal repeats (ITRs). The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell. Suitable transgenes may be readily selected by one of skill in the art. The selection of the transgene is not considered to be a limitation of this invention.

The host cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, 293 cells (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc. The most desirable cells do not carry any adenovirus gene other than E1, E2a and/or E4 ORF6; nor do they contain any other virus gene which could result in homologous recombination of a contaminating virus during the production of rAAV; and it is capable of infection or transfection of DNA and expression of the transfected DNA. In some aspects, the host cell is one that has rep and cap stably transfected in the cell.

Vector delivery to a host cell may be accomplished through various methodologies, e.g., naked DNA, a plasmid, phage, transposon, cosmid, episome, a protein in a non-viral delivery vehicle (e.g., a lipid-based carrier), virus, etc. which transfer the sequences carried thereon. The selected vector may be delivered by any suitable method, including transfection, electroporation, liposome delivery, membrane fusion techniques, high velocity DNA-coated pellets, viral infection and protoplast fusion. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Introduction of the molecules (as plasmids or viruses) into the host cell may also be accomplished using techniques known to the skilled artisan.

The rAAV may comprise a capsid serotype selected from the group consisting of AAV1, AAV2, AAV3, AAV4, AAV5, AAV6, AAV7, AAV8, AAV9, AAV10, AAV11, AAV12, AAV13, AAV2.5, AAV-DJ, AAV-DJ/8, AAV-Rh10, AAV-retro, AAV-PHP.B, AAV8-PHP.eB, AAV-PHP.S and others. In other aspects, the rAAV may comprise a capsid serotype derived from the group consisting of AAV1, AAV2, AAV3, AAV4, AAV5, AAV6, AAV7, AAV8, AAV9, AAV10, AAV11, AAV12, AAV13, AAV2.5, AAV-DJ, AAV-DJ/8, AAV-Rh10, AAV-retro, AAV-PHP.B, AAV8-PHP.eB, AAV-PHP.S and others. In some aspects, the rAAV may comprise a variant of a capsid serotype selected from the group consisting of AAV1, AAV2, AAV3, AAV4, AAV5, AAV6, AAV7, AAV8, AAV9, AAV10, AAV11, AAV12, AAV13, AAV2.5, AAV-DJ, AAV-DJ/8, AAV-Rh10, AAV-retro, AAV-PHP.B, AAV8-PHP.eB, AAV-PHP.S and others. In some aspects, the rAAV may comprise an ancestral AAV capsid serotype.

In other aspects, the rAAV may comprise a tissue specific capsid serotype. For example, in some aspects, the rAAV may comprise a muscle specific capsid serotype selected from the group consisting of AAVmyo, AAVmyo2, AAVmyo3, myoAAV-1A, myoAAV-2A, myoAAV-3A, myoAAV-4A, AAV587MTP, AAVM41, AAV9.45, and AAV9.61. In other aspects, the rAAV may comprise a lung specific capsid serotype selected from the group consisting of AAV1, AAV2-ESGHGYF, AAV2-ESGHGYF-Y730F-T491V, AAV2-PRSADLA, AAV2-PRSTSDP, AAV2.5T, AAV2H22, AAV4, AAV5, AAV5-PK2, AAV6.2, AAV6.2FF, AAV8-Y733F, AAV9.452sub.LUNG1, HAE1, and HAE2. In other aspects, the rAAV may comprise a retina specific capsid serotype selected from the group consisting of AAV2-HBKO, AAV2-7M8, AAV2.GL, AAV2.NN, AAV44.9, AAV44.9 (E531D), AAV8BP2, AAV6-K531E-R576Q-K493S-K459S, AAV2tYF, AAV-4D-R100, AAV9.GL, AAV9.NN, and ShH10. In other aspects, the rAAV may comprise a liver specific capsid serotype selected from the group consisting of AAVhum.8, AAV-L2-10, AAV3B-DE5, AAV-KP1, AAV-DJ, AAV-DJ-K137R/T251A/S503A, AAV3-S663V-T492V, AAVrh10, Spark100, AAVrh74, and AAV-SL65. In other aspects, the rAAV may comprise a capsid serotype capable of crossing the blood-brain barrier (BBB) selected from the group consisting of AAV-PHP.B, AAV-PHP.eB, AAV-PHP.S, AAV.CAP-B10, AAV-F, AAV-S, AAV-PHP.Cs, AAV-PHP.C2, AAV 9P31, AAV-MaCPNS1,AAV-MaCPNS2, AAV.CPP.16, AAV.CPP.21, AAV9.HR, AAV-B1, AAV/Olig001, AAV-AS, AAV-BR1, AAV-ShH10, AAV-ShH10Y445F, AAV Retrograde, AAV1/2 hybrid, AAV.GTX, AAV-Cap-B22, PHP.B1, PHP.B2, PHP.B3, AAVIRX, AAV-True Type (AAV-TT), AAV-PHP. V, AAV-PHP.C, AAV-PHP.N, AAV9_A2, AAV1_P5, VCAP-101, d VCAP-102, and AAV8-THR.

In some aspects, collecting the rAAV from media comprising an AAV host cell comprises spin centrifugation to collect the host cells and discard the supernatant. In situ lysis followed by filtration and concentration by tangential flow filtration may then be utilized to clarify the whole cell lysate. In other aspects, affinity and/or ion-exchange chromatography (IEC) methods are utilized to separate the rAAV from the whole cell lysate.

In some aspects, the collected rAAV undergoes release testing, e.g. quality control, to determine the safety, purity, concentration, identity, potency, and/or stability of the rAAV product. In some aspects, variables affecting rAAV product quality comprise cell culture to vector generation productivity, and production of nonvector virus subpopulations such as rcAAVs, AAV-encapsidated residual DNA impurities and empty capsids,

Thus, provided herein, in some aspects, is a method for performing quality control on a recombinant adeno-associated virus (rAAV) product, comprising: a) producing the rAAV product; b) performing a first dPCR assay comprising the steps of: (i) serially diluting the rAAV sample; (ii) determining the level of each of a first junction of an rcAAV genome, a second junction of an rcAAV genome, and a third junction of an rcAAV genome in the rAAV sample by dPCR; (iii) identifying an optimal dilution for single dPCR quantification of rcAAV in the rAAV sample; c) performing a second dPCR assay comprising the steps of: (i) diluting the rAAV sample according to the optimal dilution for single dPCR quantification identified in step (b); (ii) determining the level of each of the first junction of the rcAAV genome, the second junction of the rcAAV genome, and the third junction of the rcAAV genome in the rAAV sample by dPCR; (iii) detecting rcAAV in the rAAV sample when the first junction of the rcAAV genome, the second junction of the rcAAV genome, and the third junction of the rcAAV genome in the rAAV sample are detected within a single partition, d) discarding the produced rAAV product when rcAAV is detectibly present or detectable above a threshold, or not-discarding the produced AAV when rcAAV is not detectibly present or detectable below a threshold; and wherein the rcAAV comprises a contiguous 5′ITR, Rep gene, Cap gene, and 3′ITR, and wherein said first junction comprises a nucleotide sequence comprising the 3′ end of the 5′ITR and the 5′ end of the Rep gene; said second junction comprises a nucleotide sequence comprising the 3′ end of the Rep gene and the 5′ end of the Cap gene; said third junction comprises a nucleotide sequence comprising the 3′ end of the Cap gene and the 5′ end of the 3′ITR.

In some aspects, the threshold is between less than 1 rcAAV genome per about 107 viral genomes to less than 1 rcAAV genome per about 109 viral genomes. In some aspects, the threshold comprises less than 1 rcAAV genome per about 107 viral genomes. In some aspects, the threshold comprises less than 1 rcAAV genome per about 108 viral genomes. In some aspects, the threshold comprises less than 1 rcAAV genome per about 109 viral genomes.

### V. Kits

In some aspects, disclosed herein are kits detecting rcAAV particles, wherein the rcAAV particles comprise a contiguous 5′ITR, Rep gene, Cap gene, and 3′ITR, the kit comprising: a. forward and reverse primers capable of hybridizing to a first junction of an rcAAV genome comprising the 3′ end of the 5′ITR and the 5′ end of the Rep gene; b. forward and reverse primers capable of hybridizing to a second junction comprising the 3′ end of the Rep gene and the 5′ end of the Cap gene; c. forward and reverse primers capable of hybridizing to a third junction comprising the comprising the 3′ end of the Cap gene and the 5′ end of the 3′ITR; and d. fluorescently labeled hydrolysis probes capable of hybridizing to the first junction, the second junction, and the third junction of the rcAAV genome. In some aspects, the kits further comprise instructions for use in accordance with the methods of this disclosure.

In some aspects, the kits further comprise positive and negative control samples. In some aspects, the positive control sample comprises an ITRrep nucleotide sequence or an ITRcap nucleotide sequence. In some aspects, the positive control sample comprises the nucleotide sequence set forth in SEQ ID NO:2. In some aspects, the ITRrep positive control sample is incorporated into a dPCR assay at a target concentration of between about 10 copies/μL to about 1000 copies/μL. In some aspects, the ITRrep positive control sample is incorporated into a dPCR assay at a target concentration of between about 5.0×10\hat{ }2 copies/μL to about 6.0×10\hat{ }2 copies/μL. In some aspects, the TRrep positive control sample is incorporated into a dPCR assay at a target concentration of about 5.7×10\hat{ }2 copies/μL. In other aspects, the positive control sample comprises the nucleotide sequence set forth in SEQ ID NO:3. In some aspects, the ITRcap positive control sample is incorporated into a dPCR assay at a target concentration of between about 10 copies/μL to about 1000 copies/μL. In some aspects, the ITRcap positive control sample is incorporated into a dPCR assay at a target concentration of between about 8.0×10\hat{ }2 copies/μL to about 9.0×10\hat{ }2 copies/μL. In some aspects, the TRcap positive control sample is incorporated into a dPCR assay at a target concentration of about 8.5×10\hat{ }2 copies/μL.

In other aspects, the positive control sample comprises a wild type (WT) AAV2 genome. In some aspects, the WT AAV2 positive control sample comprises a nucleotide sequence at least about 80%, at least about 85%, at least about 90%, at least about 95%, or at least about 99% identical to the nucleotide sequence set forth in SEQ ID NO:1. In some aspects, the WT AAV2 positive control sample comprises the nucleotide sequence set forth in SEQ ID NO:1.

In some aspects the negative control sample comprises nuclease free water or sample dilution buffer.

### EXAMPLES

The following examples further illustrate the invention but should not be construed as in any way limiting its scope. In light of the present disclosure and the general level of skill in the art, those of skill will appreciate that the following Examples are intended to be exemplary only and that numerous changes, modifications, and alterations can be employed without departing form the scope of the presently disclosed subject matter. The attached figures are meant to be considered as integral parts of the specification and description of the disclosure.

### Example 1

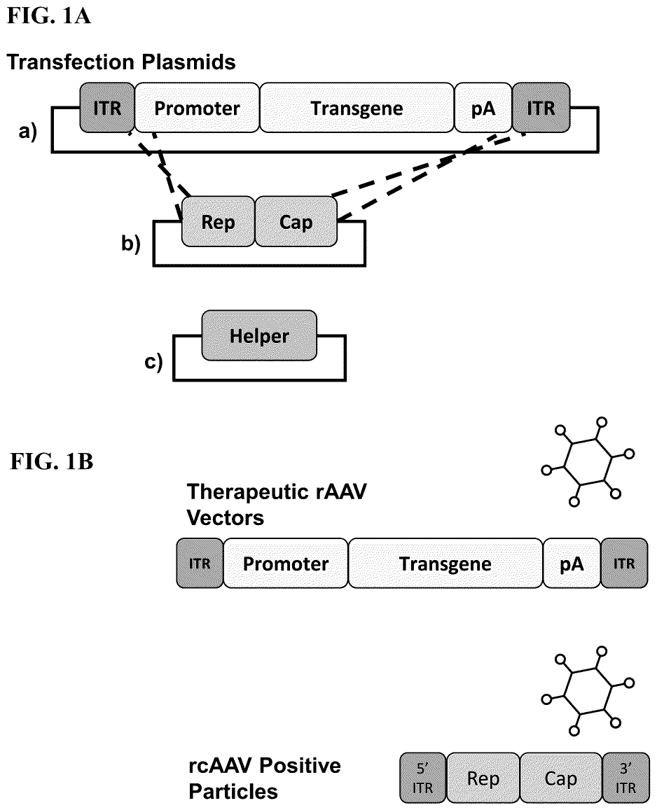

The following example describes a method for detecting and quantifying replication competent AAV (rcAAV) during the production of recombinant AAV vectors. The production of rcAAV occurs during the manufacture of rAAV in producing cells. For instance, in HEK293 cells transfected with 3 plasmids, non-homologous recombination events could lead to rcAAV generation (FIG. 1A and FIG. 1B). FIG. 1A provides a representative depiction of the structure of the transfection plasmids commonly used during rAAV production, comprising a first plasmid comprising a transgene expression construct, a second plasmid comprising AAV Rep and Cap genes, and a third plasmid comprising helper genes. A recombination event between a RepCap plasmid and a transgene expression plasmid may result in the formation of rcAAV (FIG. 1B).

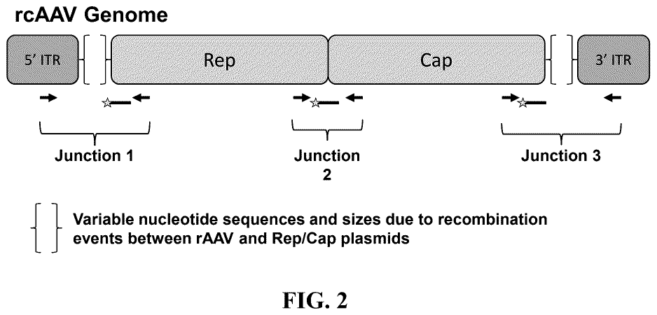

A multiplex digital PCR (dPCR) approach was used to target and identify three specific nucleotide regions within wild type AAV and rcAAVs (FIG. 2). The first nucleotide region comprised the 3′ end of the 5′ITR and the 5′ end of the Rep gene. The second nucleotide region comprised the 3′ end of the Rep gene and the 5′ end of the Cap gene. The third nucleotide junction comprised the 3′ end of the Cap gene and the 5′ end of the 3′ITR.

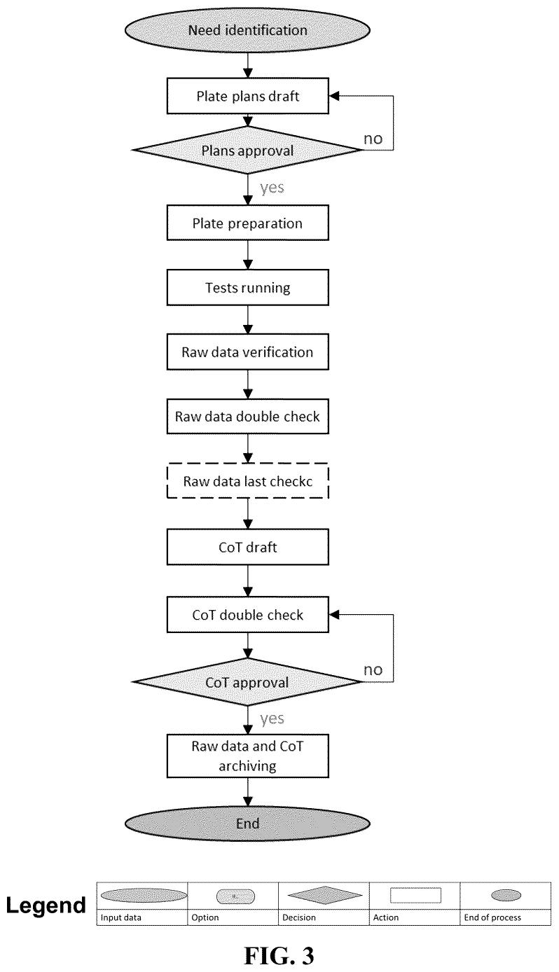

In order to accurately detect the presence of rcAAV, a 2-step dPCR assay was designed. The first dPCR assay determined the level of the 3 targeted rcAAV junctions in an rAAV sample. Based on the results generated in the first assay, an optimal dilution of the rAAV sample was determined to perform a second assay. The optimal dilution used in the second assay minimized the risk of generating false positive signals from the presence of non-linked junction fragments within the same dPCR partition. Additionally, the results from the first assay determined the number of partitions needed to achieve accurate quantification of rcAAV levels by dosing at least 100 positive partitions for the least abundant target. FIG. 3 depicts an exemplary workflow for the 2-step dPCR assay described herein.

Reagents for Assay 1 and Assay 2 are provided in Table 1.

TABLE 1
Reagents for Assay 1 and Assay 2.

| Reagents | Reference | Supplier |
|---|---|---|
| QIAcuity Probe PCR Kit | 250103 | Qiagen |
| Carrier RNA (Poly rA) 310 μg* | 1068337 | Qiagen |
| poloxamer 188 solution, 10%, | P5556 | Sigma |
| sterile | | |
| TE buffer (1x) ph 8.0 | 348619 | APPLICHEM or other |
| molecular biology grade | | suppliers |
| DPBS | 14040-117 | Gibco or other suppliers |
| Nuclease-free water | 12060346 | Invitrogen |

### Assay 1

To avoid false positive signals generated from particles containing partial rcAAV genomes, a first assay was performed to determine an optimal dilution for a given rAAV sample. Positive controls incorporated into the first assay included a nucleotide sequence comprising ITRrep (SEQ ID NO:2) at a target final concentration of 5.7×102 copies/μL, a nucleotide sequence comprising ITRcap (SEQ ID NO:3) at a target final concentration of 8.5×102 copies/μL, and a wt AAV2 vector comprising the nucleotide sequence set forth in SEQ ID NO:1. Two negative controls were also incorporated into the first assay. The first negative control comprised nuclease free water without the addition of a nucleic acid sample and the second negative control comprised sample dilution buffer without the addition of a nucleic acid sample.

The rAAV sample was serially diluted to provide a range of dPCR results used to calculate the optimal dilution of the rAAV sample for Assay 2. Samples for the first dPCR assay were prepared by mixing 26 μL of dPCR mix with 4 μL of each diluted sample and were homogenized by vortexing. Nucleotide sequences for the primers used to detect the 3 targeted rcAAV junctions are provided herein. The samples were then loaded onto a 96-well plate (12 μL per well). The plate was then placed inside a QIAcuity dPCR machine and the dPCR run was initiated using the parameters described in Table 2.

TABLE 2
dPCR thermocycling parameters

| Step | Time | | Temperature |
|---|---|---|---|
| PCR initial heat activation | 2 | min | 95° C. |

2-step cycling (40 cycles)

| | | | |
|---|---|---|---|
| Denaturation | 15 | s | 95° C. |
| Combined annealing and extension | 30 | s | 60° C. |

Imaging of Assay 1 was performed using green (FAM), yellow (HEX) and orange (TAMRA) channels. The FAM and HEX channels were imaged using an exposure time of 500 ms and a gain of 6. The TAMRA channel was imaged using an exposure time of 400 ms and a gain of 6. Analysis of Assay 1 was performed using the QIAcuity software. Based on the imaging results obtained from Assay 1, a Poisson Law calculation was employed to determine the concentration of nucleic acids comprising one or more of the rcAAV nucleotide junctions; the ITRrep junction, the the repcap junction, and/or the ITRcap junction. Nucleic acids comprising the three rcAAV junctions were identified as rcAAVs.

Results from the first assay were used to determine an optimal dilution to quantify the amount of rcAAV present in a given rAAV sample.

For accurate quantification of complete rcAAV genomes and to avoid the probability that 2 or more particles containing partial rcAAV genomes were quantified as putative rcAAV particles, sample dilution was adapted for each sample and performed in order to isolate triple positive particles from single and double harboring non-linked fragments.

Selection of the optimal dilution was based on the following criteria: a) the number of triple positive partitions (the 3 targeted fragments are supposed to be not linked or independent) should be less than about 0.1, meaning the probability to have a triple positive partitions should be less than 0.1 over the total number of partitions and b) the number of double positive partitions should be less than about 1.

The number of positive partitions identified in Assay 1 was used to determine the minimum number of runs to be employed in assay 2, calculated using Equation 1.

$$Equation 1$$

$$Minimum number of runs = \frac{[ 100 * \frac{\# partitions obtained for a dilution during assay 1}{\begin{matrix}\# positive partitions obtained for a \\ dilution during assay 1\end{matrix}} ]}{maximum \# partitions per well}$$

### Assay 2

For the second assay, the recombinant AAV sample was diluted in SDB using the optimal dilution identified in Assay 1. The number of runs to be conducted (i.e., the number of wells to prepare) was also determined in Assay 1. The same positive and negative controls tested in Assay 1 were utilized for Assay 2. To avoid contamination of the rAAV sample in Assay 2, positive and negative control dPCR reactions were run after the dPCR rAAV sample reaction.

The optimal dilution of the rAAV sample was mixed with dPCR mix as described above. The samples were then homogenized by vortexing and loaded onto a 96-well plate (12 μL per well). Assay 2 utilized the same dPCR parameters identified in Assay 1 (see Table 2). Imaging and analysis of the dPCR reaction was conducted in the same manner as Assay 1, above.

Acceptance criteria for the positive controls, WT AAV2, ITRrep nucleic acid, and ITRcap nucleic acid are provided in Table 3.

TABLE 3
Positive control acceptance criteria.

| | Titer copies/mL | | | |
|---|---|---|---|---|
| | | | | Triple |
| Test Item | ITRrep | ITRcap | REPCAP | Positive |
| WT AAV2 | 4.0E12- | 4.0E12- | 9.0E12- | 1.0E+12- |
| | 1E13 | 1.1E13 | 2.3E13 | 2.6E12 |
| ITRrep nucleic acid | NA | NA | NA | NA |
| ITRcap nucleic acid | NA | NA | NA | NA |

Acceptance criteria for the negative controls are provided in Table 4.

TABLE 4
Negative control acceptance criteria.

| | Copies/well | | | |
|---|---|---|---|---|
| Test Item | ITRrep | ITRcap | REPCAP | Triple Positive |
| Water | <3 | <3 | <3 | 0 |
| Dilution buffer | <3 | <3 | <3 | 0 |

### Example 2

The following example describes the detection and quantification of rcAAV within various rAAV production samples.

rAAV samples were collected during the production of rAAV-T1 (AAV2) and the method of detecting rcAAV was carried out according to the methods described in Example 1. The rAAV-T1 vector comprised a promoter, transgene 1, and a polyA sequence flanked by ITR sequences (FIG. 1A).

To test the influence of production size on rcAAV formation, rAAV-T1 was produced in the AAV2/2x serotype in batch sizes of 10 L, 50 L, and 250 L, and the levels of rcAAV formation were quantified. To test the influence of AAV serotype on rcAAV production, rAAV samples were produced in the AAV serotypes AAV2/2x, AAV2/8, and AAV2/9 and the levels of rcAAV formation were quantified. The levels of rcAAV formation during AAV production were also compared between plasmid DNA and synthetic DNA. To test the influence of the rAAV-T1 payload sequences on rcAAV formation, synthetic DNA comprising the rAAV-T1 (AAV2/2x) payload or the rAAV-T1 (AAV2/8) payload were produced and the levels of rcAAV formation were tested. As a positive control, wild type AAV2/2x (wt AAV2/2x) and wild type AAV8 (wt AAV2/8) were tested, as wt AAV and rcAAV comprise similar genomes and wt AAV are replication competent.

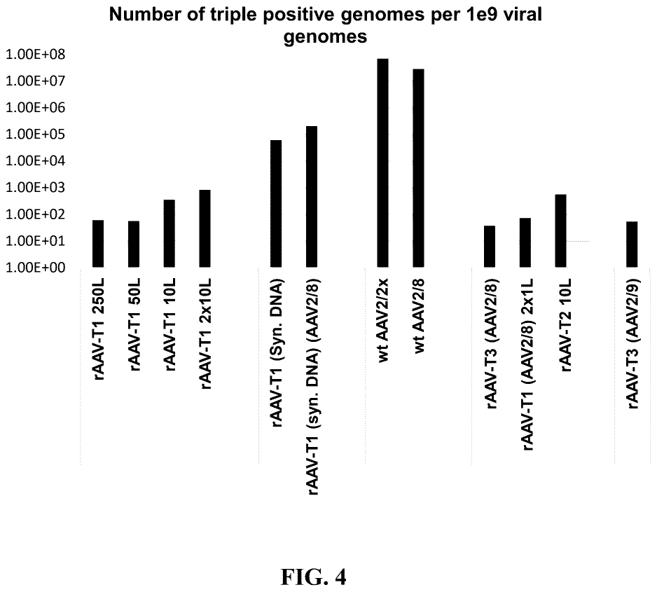

Results from the analysis of rcAAV formation during rAAV production are shown in FIG. 4 and Table 5. Manufacturing rAAV (serotype 2, 8 and 9) with plasmid DNA leads to 1×10¹ to 1×10³ triple positive (i.e., rcAAV) copies per 1×109 viral genomes (vg). The formation of rcAAV was elevated when using synthetic DNA when compared to the levels of rcAAV formation when using plasmid DNA. Additionally, formation of rcAAV was reduced when rAAV was produced in large volumes. The amount of rcAAV detected when rAAV-T1 produced in 10L was between 4×10⁻⁷ to 8×10⁻⁷ copies/vg, while the amount of rcAAV detected when rAAV-T1 produced in 250 L was 1.0×10⁻⁷ copies/vg.

These results demonstrate that the method of detecting rcAAV described herein can assess rcAAV formation during the production of rAAV at industrial scale.

TABLE 5
Detection and quantification of rcAAV during rAAV production.

| | | Vg | Infectious | | | | |
|---|---|---|---|---|---|---|---|
| | Vector | Titer | Titer | Titer copies/mL (copies/vg) | | | |
| AAV Sample | Preparation | (vg/mL) | (ip/mL) | ITRrep | ITRcap | REPCAP | Triple Pos. |
| rAAV-T1 | rAAV-T1 | 4.8E+12 | / | (0.0001%) | (0.002%) | (0.1%) | 1.00E−07 |
| (AAV2/2×) | 250 L | | | | | | |
| | rAAV- | 4.7E+12 | / | (0.0001%) | (0.002%) | (0.1%) | 1.00E−07 |
| | T150 L | | | | | | |
| | rAAV-T1 | 4.9E+12 | / | (0.002%) | (0.005%) | (0.1%) | 4.00E−07 |
| | 10 L 3rd | | | | | | |
| | rAAV-T1 | 5.6E+12 | / | (0.002%) | (0.05%) | (0.1%) | 8.00E−07 |
| | 10 L | | | | | | |
| | 2 × 10 L | | | | | | |
| syntheticDNA | rAAV-T1 | 3.5E+10 | / | (0.02%) | (0.03%) | (0.4%) | 4.00E−05 |
| | synthetic | | | | | | |
| | DNA | | | | | | |
| | rAAV-T1 | 1.7E+11 | / | (0.06%) | (0.04%) | (0.5%) | 2.00E−04 |
| | (AAV8) | | | | | | |
| | synthetic | | | | | | |
| | DNA | | | | | | |
| WTAAV | wtAAV2/2× | 2.10E+12 | 2.73E+10 | (43%) | (49%) | (100%) | (6.6%) |
| | wtAAV2/8 | 3.10E+12 | 1.75E+07 | (47%) | (47%) | (100%) | (2.8%) |
| AAV8 | rAAV-T3 | 8.00E+12 | / | (0.001%) | (0.002%) | (0.02%) | 3.69E−08 |
| | (AAV2/8) | | | | | | |
| | rAAV-T1 | 5.40E+12 | / | (0.0001%) | (0.001%) | (0.1%) | 7.00E−08 |
| | 2 × 1 L | | | | | | |
| | rAAV-T2 | 2.40E+12 | / | (0.0008%) | (0.009%) | (0.7%) | 5.50E−07 |
| | 10 L | | | | | | |
| AAV9 | rAAV-T3 | 2.10E+12 | / | (0.002%) | (0.003%) | (0.027%) | 5.3E−08 |
| | (AAV2/9) | | | | | | |

The method of detecting rcAAV described herein was further compared to a cell-based assay routinely used in the field to detect rcAAV. The cell-based approach is the industry standard approach for detecting rcAAV during rAAV manufacturing. After one to three rounds of amplification, a quantitative PCR (qPCR) analysis is performed to detect the presence of Rep (+/− Cap). The method uses engineered or WT positive controls (10-20 IU per flask) and a suitability testing is performed to check for product interference. The method can determine if rcAAVs are present above a certain level, but cannot determine the exact quantity of rcAAVs in the vector batch. Limitations of this cell-based approach to detecting rcAAV are provided in Table 6

TABLE 6
Limitations of cell-based assays for rcAAV detection.

| Variable | Detail | Result |
|---|---|---|
| AAV serotype | Contrary to in-vivo, AAV2 is 100 to 1000-fold more | Assay sensitivity |
| | infectious than AAV8 in cultured cells | is serotype- |
| | | dependent |
| Cell line used | HEK293 cells are most commonly used, but contract | Assay sensitivity |
| | research orginizations (CROs) may utilize differing cell | is cell-line |
| | lines for AAV production | dependent |

Detection of Rep2 or Rep2/CapX

| | | |
|---|---|---|
| Endpoint Analysis | Positivity defined at round 3: limit of detection (LOD) | Endpoint analysis |
| | based on copies/reaction or copies/cell (depending on | and LOD are |
| | the CRO) | different |
| Viral genome load | Viral genome titers vary among CROs/CDMO | Different vg loads |
| | | can lead to |
| | | different results |
| Positive control | Determination of infectivity of positive control can vary | Bias in assay |
| | between CROs due to a difference in quantification | sensitivity |
| | method | |

To test whether the cell-based assay could match the sensitivity of the dPCR-approach described herein, rcAAV particles were added to the cell-based assay at levels matching those quantified during the production of rAAV measured above. The amount of rcAAV particles added to the cell-based assay are shown in Table 7.

TABLE 7
Cell-based assay to detect rcAAV.

| | | Number of | | | |
|---|---|---|---|---|---|
| | | rcAAV | P1 (1E9 | P2 (1E9 | P3 (1E9 |
| | | particles | vg for test | vg for test | vg for test |
| | | added to the | sample | sample | sample |
| AAV | Vector | cell-based | and 10 ip | and 10 ip | and 10 ip |
| Sample | Preparation | assay | for WTAAV) | for WTAAV) | for WTAAV) |
| rAAV-T1 | rAAV-T1 250 L | 60 (worst case) | −/− | +/+ | +/+ |
| (AAV2/2x) | | in 1E9 vg | | | |
| | | (Pos at 1E8 vg) | | | |
| | rAAV-T1 50 L | 55 (worst case) | −/− | −/+ | −/+ |
| | | in 1E9 vg | | | |
| | | (Neg at 1E8vg) | | | |
| | rAAV-T1 10 L | 351 (worst case) | −/− | +/+ | +/+ |
| | | in 1E9 vg | | | |
| | | (Neg at 1E8 vg) | | | |
| | rAAV-T1 10 L | 826 (worst case) | | +/+/+ | +/+/+ |
| | 2 × 10 L | in 1E9 vg | | | |
| | | (Pos at 1E8 vg) | +/+/+ (small | | |
| | | | positive signal) | | |
| Synthetic | rAAV-T1 | 6E+04 (worst case) | − | + | + |
| DNA | | in 1E9vg | | | |
| | rAAV-T1 | 2E+05 (worst case) | − | − | − |
| | (AAV2/8) | in 1E9vg | | | |
| wtAAV | wtAAV2/2x | 50 (worst case) | + | + | + |
| | | (10 ip) | | | |
| | wtAAV2/8 | 5E+04 (10 ip) | + (small | + | + |
| | | | positive signal) | | |
| AAV8 | rAAV-T3 | 37 (worst case) | Not Done | Not Done | −/− |
| | (AAV2/8) | in 1E9vg | | | |
| | rAAV-T1 | 72 (worst case) | Not Done | Not Done | −/− |
| | 2 × 1 L | in 1E9vg | | | |
| | rAAV-T2 10 L | 550 (worst case) | Not Done | Not Done | −/− |
| | | in 1E9vg | | | |
| AAV9 | rAAV-T3 | 53 (worst case) | Not Done | Not Done | −/− |
| | (AAV2/9) | in 1E9vg | | | |

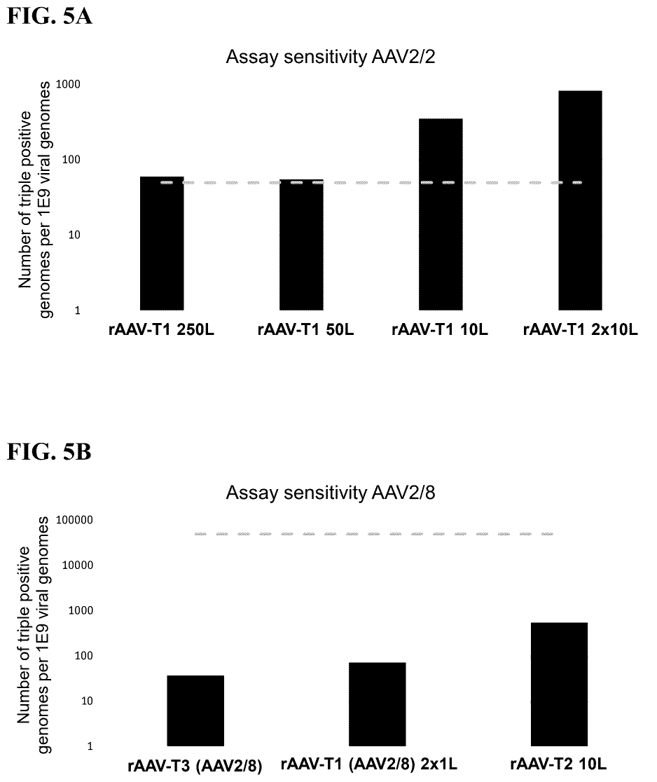

These results show that the limit of detection for the cell-based assay was 10 infectious particles (IP) for engineered (WT) AAV2/2x and (WT) AAV2/8, which was at or above the number of rcAAV particles detected during rAAV production using the dPCR approach described herein (FIG. 5A and FIG. 5B). 10 IP correspond to 5×101 and 5×104 Triple Positive genomes (TPg) for AAV2/2x and AAV2/8 respectively. This difference results in a lower sensitivity of the cell-based assay for detecting rAAV2/8 compared to rAAV2/2x (1000-fold less sensitive).

Additional primers were designed to amplify and detect rcAAV particles from AAV serotypes 2, 5, 8, and 9 (Table 8).

TABLE 8
Serotype specific primers.

| Serotype | Target | ID | SEQ ID NO: |
|---|---|---|---|
| Serotypes 2, 5, 8, 9 | Junction 1 | Forward primer ITRrep | 16 |
| | | Reverse primer ITRrep | 17 |
| | | Probe ITRrep | 18 |
| Serotype 2 | Junction 3 | Forward primer ITRcap | 19 |
| | | Reverse primer ITRcap | 20 |
| | | Probe ITRcap | 21 |
| | Junction 2 | Forward primer repcap | 22 |
| | | Reverse primer repcap | 23 |
| | | Probe repcap | 24 |
| Serotype 5 | Junction 3 | Forward primer ITRcap | 25 |
| | | Reverse primer ITRcap | 26 |
| | | Probe ITRcap | 27 |
| | Junction 2 | Forward primer repcap | 28 |
| | | Reverse primer repcap | 29 |
| | | Probe repcap | 30 |
| Serotype 8 | Junction 3 | Forward primer ITRcap | 31 |
| | | Reverse primer ITRcap | 32 |
| | | Probe ITRcap | 33 |
| | Junction 2 | Forward primer repcap | 34 |
| | | Reverse primer repcap | 35 |
| | | Probe repcap | 36 |
| Serotype 9 | Junction 3 | Forward primer ITRcap | 37 |
| | | Reverse primer ITRcap | 38 |
| | | Probe ITRcap | 39 |
| | Junction 2 | Forward primer repcap | 40 |
| | | Reverse primer repcap | 41 |
| | | Probe repcap | 42 |

These results demonstrate that the dPCR approach described herein comprised improved sensitivity for detecting rcAAV particles compared to the commonly used cell-based assays currently employed in the field.

SEQUENCES

| Nickname | Residues | SEQ ID |
|---|---|---|
| WT AAV2 | TTGGCCACTCCCTCTCTGCGCGCTCGCTCGCTCACTGAG | SEQ ID NO: 1 |
| (NCBI Ref. | GCCGGGCGACCAAAGGTCGCCCGACGCCCGGGCTTTGCC | |
| Sequence: | CGGGCGGCCTCAGTGAGCGAGCGAGCGCGCAGAGAGGGA | |
| NC_001401.2) | GTGGCCAACTCCATCACTAGGGGTTCCTGGAGGGGTGGA | |
| | GTCGTGACGTGAATTACGTCATAGGGTTAGGGAGGTCCT | |
| | GTATTAGAGGTCACGTGAGTGTTTTGCGACATTTTGCGA | |
| | CACCATGTGGTCACGCTGGGTATTTAAGCCCGAGTGAGC | |
| | ACGCAGGGTCTCCATTTTGAAGCGGGAGGTTTGAACGCG | |
| | CAGCCGCCATGCCGGGGTTTTACGAGATTGTGATTAAGG | |
| | TCCCCAGCGACCTTGACGAGCATCTGCCCGGCATTTCTG | |
| | ACAGCTTTGTGAACTGGGTGGCCGAGAAGGAATGGGAGT | |
| | TGCCGCCAGATTCTGACATGGATCTGAATCTGATTGAGC | |
| | AGGCACCCCTGACCGTGGCCGAGAAGCTGCAGCGCGACT | |
| | TTCTGACGGAATGGCGCCGTGTGAGTAAGGCCCCGGAGG | |
| | CCCTTTTCTTTGTGCAATTTGAGAAGGGAGAGAGCTACT | |
| | TCCACATGCACGTGCTCGTGGAAACCACCGGGGTGAAAT | |
| | CCATGGTTTTGGGACGTTTCCTGAGTCAGATTCGCGAAA | |
| | AACTGATTCAGAGAATTTACCGCGGGATCGAGCCGACTT | |
| | TGCCAAACTGGTTCGCGGTCACAAAGACCAGAAATGGCG | |
| | CCGGAGGCGGGAACAAGGTGGTGGATGAGTGCTACATCC | |
| | CCAATTACTTGCTCCCCAAAACCCAGCCTGAGCTCCAGT | |
| | GGGCGTGGACTAATATGGAACAGTATTTAAGCGCCTGTT | |
| | TGAATCTCACGGAGCGTAAACGGTTGGTGGCGCAGCATC | |
| | TGACGCACGTGTCGCAGACGCAGGAGCAGAACAAAGAGA | |
| | ATCAGAATCCCAATTCTGATGCGCCGGTGATCAGATCAA | |
| | AAACTTCAGCCAGGTACATGGAGCTGGTCGGGTGGCTCG | |
| | TGGACAAGGGGATTACCTCGGAGAAGCAGTGGATCCAGG | |
| | AGGACCAGGCCTCATACATCTCCTTCAATGCGGCCTCCA | |
| | ACTCGCGGTCCCAAATCAAGGCTGCCTTGGACAATGCGG | |
| | GAAAGATTATGAGCCTGACTAAAACCGCCCCCGACTACC | |
| | TGGTGGGCCAGCAGCCCGTGGAGGACATTTCCAGCAATC | |
| | GGATTTATAAAATTTTGGAACTAAACGGGTACGATCCCC | |
| | AATATGCGGCTTCCGTCTTTCTGGGATGGGCCACGAAAA | |
| | AGTTCGGCAAGAGGAACACCATCTGGCTGTTTGGGCCTG | |
| | CAACTACCGGGAAGACCAACATCGCGGAGGCCATAGCCC | |
| | ACACTGTGCCCTTCTACGGGTGCGTAAACTGGACCAATG | |
| | AGAACTTTCCCTTCAACGACTGTGTCGACAAGATGGTGA | |
| | TCTGGTGGGAGGAGGGGAAGATGACCGCCAAGGTCGTGG | |
| | AGTCGGCCAAAGCCATTCTCGGAGGAAGCAAGGTGCGCG | |
| | TGGACCAGAAATGCAAGTCCTCGGCCCAGATAGACCCGA | |
| | CTCCCGTGATCGTCACCTCCAACACCAACATGTGCGCCG | |
| | TGATTGACGGGAACTCAACGACCTTCGAACACCAGCAGC | |
| | CGTTGCAAGACCGGATGTTCAAATTTGAACTCACCCGCC | |
| | GTCTGGATCATGACTTTGGGAAGGTCACCAAGCAGGAAG | |
| | TCAAAGACTTTTTCCGGTGGGCAAAGGATCACGTGGTTG | |
| | AGGTGGAGCATGAATTCTACGTCAAAAAGGGTGGAGCCA | |
| | AGAAAAGACCCGCCCCCAGTGACGCAGATATAAGTGAGC | |
| | CCAAACGGGTGCGCGAGTCAGTTGCGCAGCCATCGACGT | |
| | CAGACGCGGAAGCTTCGATCAACTACGCAGACAGGTACC | |
| | AAAACAAATGTTCTCGTCACGTGGGCATGAATCTGATGC | |
| | TGTTTCCCTGCAGACAATGCGAGAGAATGAATCAGAATT | |
| | CAAATATCTGCTTCACTCACGGACAGAAAGACTGTTTAG | |
| | AGTGCTTTCCCGTGTCAGAATCTCAACCCGTTTCTGTCG | |
| | TCAAAAAGGCGTATCAGAAACTGTGCTACATTCATCATA | |
| | TCATGGGAAAGGTGCCAGACGCTTGCACTGCCTGCGATC | |
| | TGGTCAATGTGGATTTGGATGACTGCATCTTTGAACAAT | |
| | AAATGATTTAAATCAGGTATGGCTGCCGATGGTTATCTT | |
| | CCAGATTGGCTCGAGGACACTCTCTCTGAAGGAATAAGA | |
| | CAGTGGTGGAAGCTCAAACCTGGCCCACCACCACCAAAG | |
| | CCCGCAGAGCGGCATAAGGACGACAGCAGGGGTCTTGTG | |
| | CTTCCTGGGTACAAGTACCTCGGACCCTTCAACGGACTC | |
| | GACAAGGGAGAGCCGGTCAACGAGGCAGACGCCGCGGCC | |
| | CTCGAGCACGACAAAGCCTACGACCGGCAGCTCGACAGC | |
| | GGAGACAACCCGTACCTCAAGTACAACCACGCCGACGCG | |
| | GAGTTTCAGGAGCGCCTTAAAGAAGATACGTCTTTTGGG | |
| | GGCAACCTCGGACGAGCAGTCTTCCAGGCGAAAAAGAGG | |
| | GTTCTTGAACCTCTGGGCCTGGTTGAGGAACCTGTTAAG | |
| | ACGGCTCCGGGAAAAAAGAGGCCGGTAGAGCACTCTCCT | |
| | GTGGAGCCAGACTCCTCCTCGGGAACCGGAAAGGCGGGC | |
| | CAGCAGCCTGCAAGAAAAAGATTGAATTTTGGTCAGACT | |
| | GGAGACGCAGACTCAGTACCTGACCCCCAGCCTCTCGGA | |
| | CAGCCACCAGCAGCCCCCTCTGGTCTGGGAACTAATACG | |
| | ATGGCTACAGGCAGTGGCGCACCAATGGCAGACAATAAC | |
| | GAGGGCGCCGACGGAGTGGGTAATTCCTCGGGAAATTGG | |
| | CATTGCGATTCCACATGGATGGGCGACAGAGTCATCACC | |
| | ACCAGCACCCGAACCTGGGCCCTGCCCACCTACAACAAC | |
| | CACCTCTACAAACAAATTTCCAGCCAATCAGGAGCCTCG | |
| | AACGACAATCACTACTTTGGCTACAGCACCCCTTGGGGG | |
| | TATTTTGACTTCAACAGATTCCACTGCCACTTTTCACCA | |
| | CGTGACTGGCAAAGACTCATCAACAACAACTGGGGATTC | |
| | CGACCCAAGAGACTCAACTTCAAGCTCTTTAACATTCAA | |
| | GTCAAAGAGGTCACGCAGAATGACGGTACGACGACGATT | |
| | GCCAATAACCTTACCAGCACGGTTCAGGTGTTTACTGAC | |
| | TCGGAGTACCAGCTCCCGTACGTCCTCGGCTCGGCGCAT | |
| | CAAGGATGCCTCCCGCCGTTCCCAGCAGACGTCTTCATG | |
| | GTGCCACAGTATGGATACCTCACCCTGAACAACGGGAGT | |
| | CAGGCAGTAGGACGCTCTTCATTTTACTGCCTGGAGTAC | |
| | TTTCCTTCTCAGATGCTGCGTACCGGAAACAACTTTACC | |
| | TTCAGCTACACTTTTGAGGACGTTCCTTTCCACAGCAGC | |
| | TACGCTCACAGCCAGAGTCTGGACCGTCTCATGAATCCT | |
| | CTCATCGACCAGTACCTGTATTACTTGAGCAGAACAAAC | |
| | ACTCCAAGTGGAACCACCACGCAGTCAAGGCTTCAGTTT | |
| | TCTCAGGCCGGAGCGAGTGACATTCGGGACCAGTCTAGG | |
| | AACTGGCTTCCTGGACCCTGTTACCGCCAGCAGCGAGTA | |
| | TCAAAGACATCTGCGGATAACAACAACAGTGAATACTCG | |
| | TGGACTGGAGCTACCAAGTACCACCTCAATGGCAGAGAC | |
| | TCTCTGGTGAATCCGGGCCCGGCCATGGCAAGCCACAAG | |
| | GACGATGAAGAAAAGTTTTTTCCTCAGAGCGGGGTTCTC | |
| | ATCTTTGGGAAGCAAGGCTCAGAGAAAACAAATGTGGAC | |
| | ATTGAAAAGGTCATGATTACAGACGAAGAGGAAATCAGG | |
| | ACAACCAATCCCGTGGCTACGGAGCAGTATGGTTCTGTA | |
| | TCTACCAACCTCCAGAGAGGCAACAGACAAGCAGCTACC | |
| | GCAGATGTCAACACACAAGGCGTTCTTCCAGGCATGGTC | |
| | TGGCAGGACAGAGATGTGTACCTTCAGGGGCCCATCTGG | |
| | GCAAAGATTCCACACACGGACGGACATTTTCACCCCTCT | |
| | CCCCTCATGGGTGGATTCGGACTTAAACACCCTCCTCCA | |
| | CAGATTCTCATCAAGAACACCCCGGTACCTGCGAATCCT | |
| | TCGACCACCTTCAGTGCGGCAAAGTTTGCTTCCTTCATC | |
| | ACACAGTACTCCACGGGACAGGTCAGCGTGGAGATCGAG | |
| | TGGGAGCTGCAGAAGGAAAACAGCAAACGCTGGAATCCC | |
| | GAAATTCAGTACACTTCCAACTACAACAAGTCTGTTAAT | |
| | GTGGACTTTACTGTGGACACTAATGGCGTGTATTCAGAG | |
| | CCTCGCCCCATTGGCACCAGATACCTGACTCGTAATCTG | |
| | TAATTGCTTGTTAATCAATAAACCGTTTAATTCGTTTCA | |
| | GTTGAACTTTGGTCTCTGCGTATTTCTTTCTTATCTAGT | |
| | TTCCATGGCTACGTAGATAAGTAGCATGGCGGGTTAATC | |
| | ATTAACTACAAGGAACCCCTAGTGATGGAGTTGGCCACT | |
| | CCCTCTCTGCGCGCTCGCTCGCTCACTGAGGCCGGGCGA | |
| | CCAAAGGTCGCCCGACGCCCGGGCTTTGCCCGGGCGGCC | |
| | TCAGTGAGCGAGCGAGCGCGCAGAGAGGGAGTGGCCAA | |
| Gblock ITRrep | TGACCGCGAACCAGTTTGGCAAAGTCGGCTCGATCCCGC | SEQ ID NO: 2 |
| | GGTAAATTCTCTGAATCAGTTTTTCGCGAATCTGACTCA | |
| | GGAAACGTCCCAAAACCATGGATTTCACCCCGGTGGTTT | |
| | CCACGAGCACGTGCATGTGGAAGTAGCTCTCTCCCTTCT | |
| | CAAATTGCACAAAGAAAAGAGCCTCCGGGGCCTTACTCA | |
| | CACGGCGCCATTCCGTCAGAAAGTCGCGCTGCAGCTTCT | |
| | CGGCCACGGTCAGGGGTGCCTGCTCAATCAGATTCAGAT | |
| | CCATGTCAGAATCTGGCGGCAACTCCCATTCCTTCTCGG | |
| | CCACCCAGTTCACAAAGCTGTCAGAAATGCCGGGCAGAT | |
| | GCTCGTCAAGGTCGCTGGGGACCTTAATCACAATCTCGT | |
| | AAAACCCCGGCATAGGAACCCCTAGTGATGGAGTTGGCC | |
| | ACTCCCTCTCTGCGCGCTCGCTCGCTCACTGAGGCCGGG | |
| | CGACCAAAGGTC | |
| Gblock ITRcap | GGGCGACCTTTGGTCGCCCGGCCTCAGTGAGCGAGCGAG | SEQ ID NO: 3 |
| | CGCGCAGAGAGGGAGTGGCCAACTCCATCACTAGGGGTT | |
| | CCTTCTAGACAACTTTGTATAGAAAAGTTGGGAGGAAAT | |
| | GGAGACCCTGCGTGCTCACTCGGGCTTAAATACCCAGCG | |
| | TGACCACATGGTGTCGCAAAATGTCGCAAAACACTCACG | |
| | TGACCTCTAATACAGGACCTCTAGTCCTGCAGGTTTAAA | |
| | CGAATTCGCCCTTCGCAGAGACCAAAGTTCAACTGAAAC | |
| | GAATCAACCGGTTTATTGATTAACAGGCAATTACAGATT | |
| | ACGAGTCAGGTATCTGGTGCCAATGGGGCGAGGCTCTGA | |
| | ATACACGCCATTAGTGTCCACAGTAAAGTCCACATTAAC | |
| | AGACTTGTTGTAGTTGGAAGTGTACTGAATTTCGGGATT | |
| | CCAGCGTTTGCTGTTTTCCTTCTGCAGCTCCCACTCGAT | |
| | CTCCACGCTGAC | |
| Primer ITRrep | AGTGGCCAACTCCATCACTAG | SEQ ID NO: 4 |
| (Forward) | | |
| Primer ITRcap | AGTGGCCAACTCCATCACTAG | SEQ ID NO: 5 |
| (Reverse) | | |
| Primer ITRrep | GCCACCCAGTTCACAAAGC | SEQ ID NO: 6 |
| (Reverse) | | |
| Primer ITRcap | GTGGACACTAATGGCGTGTATT | SEQ ID NO: 7 |
| (Forward) | | |
| Primer repcap | GAGCTTCCACCACTGTCTTATT | SEQ ID NO: 8 |
| (Forward) | | |
| Primer repcap | GGATTTGGATGACTGCATCTTTG | SEQ ID NO: 9 |
| (Reverse) | | |
| Probe ITRrep | AGCGACCTTGACGAGCATCTGCCCGGC | SEQ ID NO: 10 |
| (FAM; BHQ-1) | | |
| Probe ITRcap | CCCATTGGCACCAGATACCTGACT | SEQ ID NO: 11 |
| (HEX; BHQ-1) | | |
| Probe repcap | TGGAAGATAACCATCGGCAGCCAT | SEQ ID NO: 12 |
| (TAMRA; BHQ-2) | | |
| rcAAV | AGTGGCCAACTCCATCACTAGGGGTTCCTGGAGGGGTGG | SEQ ID NO: 13 |
| Junction 1 | AGTCGTGACGTGAATTACGTCATAGGGTTAGGGAGGTCC | |
| (5'ITR-REP) | TGTATTAGAGGTCACGTGAGTGTTTTGCGACATTTTGCG | |
| | ACACCATGTGGTCACGCTGGGTATTTAAGCCCGAGTGAG | |
| | CACGCAGGGTCTCCATTTTGAAGCGGGAGGTTTGAACGC | |
| | GCAGCCGCCATGCCGGGGTTTTACGAGATTGTGATTAAG | |
| | GTCCCCAGCGACCTTGACGAGCATCTGCCCGGCATTTCT | |
| | GACAGCTTTGTGAACTGGGTGGC | |
| rcAAV | GGATTTGGATGACTGCATCTTTGAACAATAAATGATTTA | SEQ ID NO: 14 |
| Junction 2 | AATCAGGTATGGCTGCCGATGGTTATCTTCCAGATTGGC | |
| (REP-CAP) | TCGAGGACACTCTCTCTGAAGGAATAAGACAGTGGTGGA | |
| | AGCTC | |
| rcAAV | GTGGACACTAATGGCGTGTATTCAGAGCCTCGCCCCATT | SEQ ID NO: 15 |
| Junction 3 | GGCACCAGATACCTGACTCGTAATCTGTAATTGCTTGTT | |
| (CAP-3'ITR) | AATCAATAAACCGTTTAATTCGTTTCAGTTGAACTTTGG | |
| | TCTCTGCGTATTTCTTTCTTATCTAGTTTCCATGGCTAC | |
| | GTAGATAAGTAGCATGGCGGGTTAATCATTAACTACAAG | |
| | GAACCCCTAGTGATGGAGTTGGCCACT | |
| Serotypes 2, 5, | AGTGGCCAACTCCATCACTAG | SEQ ID NO: 16 |
| 8,9 | | |
| (pITRrepF2) | | |
| Serotypes 2, 5, | | SEQ ID NO: 17 |
| 8, 9 | GCCACCCAGTTCACAAAGC | |
| (pITRrepR2) | | |
| Serotypes 2, 5, | | SEQ ID NO: 18 |
| 8, 9 | AGCGACCTTGACGAGCATCTGCCCGGC | |
| (probeITRrep2) | | |
| Serotype 2 | GTGGACACTAATGGCGTGTATT | SEQ ID NO: 19 |
| (pITRcapF2) | | |
| Serotype 2 | AGTGGCCAACTCCATCACTAG | SEQ ID NO: 20 |
| (pITRrepF2) | | |
| Serotype 2 | CCCATTGGCACCAGATACCTGACT | SEQ ID NO: 21 |
| (probeITRcap2) | | |
| Serotype 2 | GAGCTTCCACCACTGTCTTATT | SEQ ID NO: 22 |
| (prepcapF2) | | |
| Serotype 2 | GGATTTGGATGACTGCATCTTTG | SEQ ID NO: 23 |
| (prepcapR2) | | |
| Serotype 2 | TGGAAGATAACCATCGGCAGCCAT | SEQ ID NO: 24 |
| (proberepcap2) | | |
| Serotype 5 | GGGAATACAGAACCACCAGAC | SEQ ID NO: 25 |
| (pITRcapF5) | | |
| Serotype 5 | AGTGGCCAACTCCATCACTAG | SEQ ID NO: 26 |
| (pITRrepF2) | | |
| Serotype 5 | TCGGAACCCGATACCTTACCCGA | SEQ ID NO: 27 |
| (probeITRcap5) | | |
| Serotype 5 | GGATTTGGATGACTGCATCTTTG | SEQ ID NO: 28 |
| (prepcapR2) | | |
| Serotype 5 | GACCTTCACCAACTTCTTCCA | SEQ ID NO: 29 |
| (prepcapR5) | | |
| Serotype 5 | TTGTTGATCACCCTCCAGATTGGT | SEQ ID NO: 30 |
| (proberepcap5) | | |
| Serotype 8 | TCTACAAGTGTGGACTTTGCTG | SEQ ID NO: 31 |
| (pITRcapF8) | | |
| Serotype 8 | AGTGGCCAACTCCATCACTAG | SEQ ID NO: 32 |
| (pITRrepF2) | | |
| Serotype 8 | AGGCGTGTACTCTGAACCCCGCCCCATTGG | SEQ ID NO: 33 |
| (probeITRcap8) | | |
| Serotype 8 | GGATTTGGATGACTGCATCTTTG | SEQ ID NO: 34 |
| (prepcapR2) | | |
| Serotype 8 | GGGGCTCCAGGTTTCAGC | SEQ ID NO: 35 |
| (prepcapR8) | | |
| Serotype 8 | TGGAAGATAACCATCGGCAGCCAT | SEQ ID NO: 36 |
| (proberepcap2) | | |
| Serotype 9 | TGAAGGTGTATATAGTGAACCCCG | SEQ ID NO: 37 |
| (pITRcapF9) | | |
| Serotype 9 | AGTGGCCAACTCCATCACTAG | SEQ ID NO: 38 |
| (pITRrepF2) | | |
| Serotype 9 | CCCATTGGCACCAGATACCTGACT | SEQ ID NO: 39 |
| (probeITRcap2) | | |
| Serotype 9 | GGACTTGGATGACTGTGTTTCTG | SEQ ID NO: 40 |
| (prepcapF9) | | |
| Serotype 9 | CACCACTCGCGAATTCCTTC | SEQ ID NO: 41 |
| (prepcapR9) | | |
| Serotype 9 | TGGAAGATAACCATCGGCAGCCAT | SEQ ID NO: 42 |
| (proberepcap2) | | |

What is claimed is:

1. A method of detecting replication competent adeno-associated virus (rcAAV) in a recombinant adeno-associated virus (rAAV) sample, comprising at least one multiplex digital polymerase chain reaction (dPCR) amplifying at least three junctions of a rcAAV genome comprising a first junction of an rcAAV genome, a second junction of an rcAAV genome, and a third junction of an rcAAV genome in the rAAV sample,
wherein the rcAAV comprises a contiguous 5′ITR, Rep gene, Cap gene, and 3′ITR, and wherein
said first junction comprises a nucleotide sequence comprising the 3′ end of the 5′ITR and the 5′ end of the Rep gene;
said second junction comprises a nucleotide sequence comprising the 3′ end of the Rep gene and the 5′ end of the Cap gene;
said third junction comprises a nucleotide sequence comprising the 3′ end of the Cap gene and the 5′ end of the 3′ITR.

2. A method of detecting replication competent adeno-associated virus (rcAAV) in a recombinant adeno-associated virus (rAAV) sample, comprising a multiplex digital polymerase chain reaction (dPCR) comprising;
(a) a first dPCR assay comprising the steps of:
(i) serially diluting the rAAV sample;
(ii) determining the level of each of a first junction of an rcAAV genome, a second junction of an rcAAV genome, and a third junction of an rcAAV genome in the rAAV sample by dPCR;
(iii) identifying an optimal dilution for single dPCR quantification of rcAAV in the rAAV sample; and
(b) a second dPCR assay comprising the steps of:
(i) diluting the rAAV sample according to the optimal dilution for single dPCR quantification identified in step (a);
(ii) determining the level of each of the first junction of the rcAAV genome, the second junction of the rcAAV genome, and the third junction of the rcAAV genome in the rAAV sample by dPCR;
(iii) detecting rcAAV in the rAAV sample when the first junction of the rcAAV genome, the second junction of the rcAAV genome, and the third junction of the rcAAV genome in the rAAV sample are detected within a single partition,
wherein the rcAAV comprises a contiguous 5′ITR, Rep gene, Cap gene, and 3′ITR, and wherein
said first junction comprises a nucleotide sequence comprising the 3′ end of the 5′ITR and the 5′ end of the Rep gene;
said second junction comprises a nucleotide sequence comprising the 3′ end of the Rep gene and the 5′ end of the Cap gene;
said third junction comprises a nucleotide sequence comprising the 3′ end of the Cap gene and the 5′ end of the 3′ITR.

3. The method of claim 2, wherein the optimal dilution for single dPCR quantification of rcAAV in the rAAV sample is selected as the largest dilution of the rAAV sample performed in the first dPCR assay satisfying the following criteria;
(a) the number of false triple positive partitions comprises less than about 0.1% of particles, and
(b) the number of false double positive partitions comprises less than about 1% of particles.

4. The method of claim 2, wherein a minimum number of runs to be employed in the second dPCR assay is determined using the formula:
$$Minimum number of runs = \frac{[ 100 * \frac{\# partitions obtained for a dilution during assay 1}{\begin{matrix}\# positive partitions obtained for a \\ dilution during assay 1\end{matrix}} ]}{maximum \# partitions per well} .$$

5. The method of claim 2, wherein the first junction of the rcAAV genome is amplified using forward/reverse primers capable of hybridizing to nucleotides 117-412 of the AAV2 genome set forth in SEQ ID NO:13.

6. The method of claim 2, wherein the first junction of the rcAAV genome is amplified using a forward primer comprising a nucleotide sequence at least 80% identical to the nucleotide sequence set forth in SEQ ID NO:4, and a reverse primer comprising a nucleotide sequence at least 80% identical to the nucleotide sequence set forth in SEQ ID NO:6.

7. The method of claim 2, wherein the first junction of the rcAAV genome is amplified using a forward primer comprising the nucleotide sequence set forth in SEQ ID NO:4, and a reverse primer comprising the nucleotide sequence set forth in SEQ ID NO:6.

8. The method of claim 2, wherein the second junction of the rcAAV genome is amplified using forward/reverse primers capable of hybridizing to nucleotides 2156-2277 of the AAV2 genome set forth in SEQ ID NO:14.

9. The method of claim 2, wherein the second junction of the rcAAV genome is amplified using:
1) a forward primer comprising a nucleotide sequence at least 80% identical to the nucleotide sequence set forth in SEQ ID NO:8, and a reverse primer comprising a nucleotide sequence at least 80% identical to the nucleotide sequence set forth in SEQ ID NO:9,
2) a forward primer comprising a nucleotide sequence at least 80% identical to the nucleotide sequence set forth in SEQ ID NO:22, and a reverse primer comprising a nucleotide sequence at least 80% identical to the nucleotide sequence set forth in SEQ ID NO:23,
3) a forward primer comprising a nucleotide sequence at least 80% identical to the nucleotide sequence set forth in SEQ ID NO:28, and a reverse primer comprising a nucleotide sequence at least 80% identical to the nucleotide sequence set forth in SEQ ID NO:29,
4) a forward primer comprising a nucleotide sequence at least 80% identical to the nucleotide sequence set forth in SEQ ID NO:34, and a reverse primer comprising a nucleotide sequence at least 80% identical to the nucleotide sequence set forth in SEQ ID NO:35, or
5) a forward primer comprising a nucleotide sequence at least 80% identical to the nucleotide sequence set forth in SEQ ID NO:40, and a reverse primer comprising a nucleotide sequence at least 80% identical to the nucleotide sequence set forth in SEQ ID NO:41.

10. The method of claim 2, wherein the second junction of the rcAAV genome is amplified using a forward primer comprising a nucleotide sequence at least 80% identical to the nucleotide sequence set forth in SEQ ID NO:8, and a reverse primer comprising a nucleotide sequence at least 80% identical to the nucleotide sequence set forth in SEQ ID NO:9.

11. The method of claim 2, wherein the second junction of the rcAAV genome is amplified using a forward primer comprising the nucleotide sequence set forth in SEQ ID NO:8, and a reverse primer comprising the nucleotide sequence set forth in SEQ ID NO:9.

12. The method of claim 2, wherein the third junction of the rcAAV genome is amplified using forward/reverse primers capable of hybridizing to nucleotides 4342-4563 of the AAV2 genome set forth in SEQ ID NO:15.

13. The method of claim 2, wherein the third junction of the rcAAV genome is amplified using:
1) a forward primer comprising a nucleotide sequence at least 80% identical to the nucleotide sequence set forth in SEQ ID NO:7, and a reverse primer comprising a nucleotide sequence at least 80% identical to the nucleotide sequence set forth in SEQ ID NO:5,
2) a forward primer comprising a nucleotide sequence at least 80% identical to the nucleotide sequence set forth in SEQ ID NO:19, and a reverse primer comprising a nucleotide sequence at least 80% identical to the nucleotide sequence set forth in SEQ ID NO:20,
3) a forward primer comprising a nucleotide sequence at least 80% identical to the nucleotide sequence set forth in SEQ ID NO:25, and a reverse primer comprising a nucleotide sequence at least 80% identical to the nucleotide sequence set forth in SEQ ID NO:26,
4) a forward primer comprising a nucleotide sequence at least 80% identical to the nucleotide sequence set forth in SEQ ID NO:31, and a reverse primer comprising a nucleotide sequence at least 80% identical to the nucleotide sequence set forth in SEQ ID NO:32, or
5) a forward primer comprising a nucleotide sequence at least 80% identical to the nucleotide sequence set forth in SEQ ID NO:37, and a reverse primer comprising a nucleotide sequence at least 80% identical to the nucleotide sequence set forth in SEQ ID NO:38.

14. The method of claim 2, wherein the third junction of the rcAAV genome is amplified using a forward primer comprising a nucleotide sequence at least 80% identical to the nucleotide sequence set forth in SEQ ID NO:7, and a reverse primer comprising a nucleotide sequence at least 80% identical to the nucleotide sequence set forth in SEQ ID NO:5.

15. The method of claim 2, wherein the third junction of the rcAAV genome is amplified using a forward primer comprising the nucleotide sequence set forth in SEQ ID NO:7, and a reverse primer comprising the nucleotide sequence set forth in SEQ ID NO:5.

16. The method of claim 2, wherein the first junction of the rcAAV genome, the second junction of the rcAAV genome, and the third junction of the rcAAV genome are detected in the first dPCR assay and the second dPCR assay using fluorescently labeled hydrolysis probes.

17. The method of claim 16, wherein the fluorescently labeled hydrolysis probes comprise a fluorescent label selected from the group consisting of 6-Carboxyfluorescein (FAM), Tetrachlorofluorescein (TET), Hexachlorofluorescein (HEX), and Carboxytetramethyl-rhodamine (TAMRA).

18. The method of claim 2, wherein the first junction of the rcAAV genome is detected using a fluorescently labeled hydrolysis probe comprising the nucleotide sequence set forth in SEQ ID NO: 10 or SEQ ID NO:18.

19. The method of claim 2, wherein the first junction of the rcAAV genome is detected using a fluorescently labeled hydrolysis probe comprising the nucleotide sequence set forth in SEQ ID NO:10.

20. The method of claim 2, wherein the second junction of the rcAAV genome is detected using a fluorescently labeled hydrolysis probe comprising the nucleotide sequence set forth in SEQ ID NO:12, SEQ ID NO:24, SEQ ID NO:30, SEQ ID NO:36, or SEQ ID NO:42.

21. The method of claim 2, wherein the second junction of the rcAAV genome is detected using a fluorescently labeled hydrolysis probe comprising the nucleotide sequence set forth in SEQ ID NO:12.

22. The method of claim 2, wherein the third junction of the rcAAV genome is detected using a fluorescently labeled hydrolysis probe comprising the nucleotide sequence set forth in SEQ ID NO:11, SEQ ID NO:21, SEQ ID NO:27, SEQ ID NO:33, or SEQ ID NO:39.

23. The method of claim 2, wherein the third junction of the rcAAV genome is detected using a fluorescently labeled hydrolysis probe comprising the nucleotide sequence set forth in SEQ ID NO:11.

24. A method of producing a recombinant adeno-associated virus (rAAV), the method comprising
a) recombinantly producing the AAV in a culture media comprising an AAV host cell;
b) collecting the rAAV from the media of step (a); and
c) quantifying the amount of rcAAV contaminants in the sample using the method of claim 2; and
d) discarding the produced AAV when rcAAV is detectibly present, or not-discarding the produced AAV when rcAAV is not detectibly present or is detectable at a threshold.

25. A method for performing quality control on a recombinant adeno-associated virus (rAAV) product, comprising:
a) producing the rAAV product;
b) performing a first dPCR assay comprising the steps of:
(i) serially diluting the rAAV sample;
(ii) determining the level of each of a first junction of an rcAAV genome, a second junction of an rcAAV genome, and a third junction of an rcAAV genome in the rAAV sample by dPCR;
(iii) identifying an optimal dilution for single dPCR quantification of rcAAV in the rAAV sample; and
c) performing a second dPCR assay comprising the steps of:
(i) diluting the rAAV sample according to the optimal dilution for single dPCR quantification identified in step (b);
(ii) determining the level of each of the first junction of the rcAAV genome, the second junction of the rcAAV genome, and the third junction of the rcAAV genome in the rAAV sample by dPCR;
(iii) detecting rcAAV in the rAAV sample when the first junction of the rcAAV genome, the second junction of the rcAAV genome, and the third junction of the rcAAV genome in the rAAV sample are detected within a single partition,
d) discarding the produced rAAV product when rcAAV is detectibly present or detectable above a threshold, or not-discarding the produced AAV when rcAAV is not detectibly present or detectable below a threshold; and
wherein the rcAAV comprises a contiguous 5′ITR, Rep gene, Cap gene, and 3′ITR, and wherein
said first junction comprises a nucleotide sequence comprising the 3′ end of the 5′ITR and the 5′ end of the Rep gene;
said second junction comprises a nucleotide sequence comprising the 3′ end of the Rep gene and the 5′ end of the Cap gene;
said third junction comprises a nucleotide sequence comprising the 3′ end of the Cap gene and the 5′ end of the 3′ITR.

26. A kit for detecting rcAAV particles, wherein the rcAAV particles comprise a contiguous 5′ITR, Rep gene, Cap gene, and 3′ITR, the kit comprising:
e. forward and reverse primers capable of hybridizing to a first junction of an rcAAV genome comprising the 3′ end of the 5′ITR and the 5′ end of the Rep gene;
f. forward and reverse primers capable of hybridizing to a second junction comprising the 3′ end of the Rep gene and the 5′ end of the Cap gene;
g. forward and reverse primers capable of hybridizing to a third junction comprising the comprising the 3′ end of the Cap gene and the 5′ end of the 3′ITR; and
h. fluorescently labeled hydrolysis probes capable of hybridizing to the first junction, the second junction, and the third junction of the rcAAV genome.
